SEQUENCE LISTING (146 sequences)

SEQ ID NO: 1 - protein, 37 aa, synthetic construct
  MOD_RES 37: note = AMIDATION
KCNTATCATQRLANFLVHSSNNFGPILPPTNVGSNTY

SEQ ID NO: 2 - protein, 37 aa, synthetic construct
  MOD_RES 37: note = AMIDATION
KCNTATCATQRLAEFLRHSSNNFGPILPPTNVGSNTP

SEQ ID NO: 3 - protein, 37 aa, Homo sapiens [sequence omitted]

SEQ ID NO: 4 - protein, 37 aa, Rattus norvegicus
  MOD_RES 37: note = AMIDATION
KCNTATCATQRLANFLVRSSNNLGPVLPPTNVGSNTY

SEQ ID NO: 5 - skipped

SEQ ID NO: 6 - protein, 25 aa, synthetic construct
  VARIANT 1: note = X can be A, V or M
  VARIANT 2: note = X can be T or L
  VARIANT 3: note = X can be Q or G
  VARIANT 4: note = X can be R or K
  VARIANT 6: note = X can be A or S
  VARIANT 7: note = X can be any amino acid
  VARIANT 8: note = X can be E, D or F
  VARIANT 11: note = X can be K or R
  VARIANT 13: note = X can be Q or K
  VARIANT 15: note = X can be Y or F
  VARIANT 17: note = X can be R or K
  VARIANT 19: note = X can be Q, D, P, K or N
  VARIANT 20: note = X can be V or T
  VARIANT 22: note = X can be S or A
  VARIANT 23: note = X can be K, N, G or D
  VARIANT 24: note = X can be T or A
  VARIANT 25: note = X can be any amino acid
XXXXLXXXLHXLXTXPXTXXGXXXX

SEQ ID NO: 7 - protein, 6 aa, synthetic construct
ATQRLA

SEQ ID NO: 8 - protein, 11 aa, synthetic construct
ELHKLQTYPRT

SEQ ID NO: 9 - protein, 11 aa, synthetic construct
ELHKLKTYPRT

SEQ ID NO: 10 - protein, 5 aa, synthetic construct
DDDDY

SEQ ID NO: 11 - protein, 5 aa, synthetic construct
HPFHL

SEQ ID NO: 12 - protein, 33 aa, synthetic construct
  MOD_RES 33: note = AMIDATION
  MOD_RES 1: note = Type (i) lipidation
KCSNLSTCVLGKLSQELHKLQTYPRTNTGSGTY

SEQ ID NO: 13 - protein, 33 aa, synthetic construct
  MOD_RES 33: note = AMIDATION
  MOD_RES 1: note = Type (i) lipidation
KCSNLSTCVLGKLSQELHKLQTYPRTKTGSGTP

SEQ ID NO: 14 - protein, 34 aa, synthetic construct
  MOD_RES 34: note = AMIDATION
  MOD_RES 1: note = Type (i) lipidation
KKCSNLSTCVLGKLSQELHKLQTYPRTNTGSGTP

SEQ ID NO: 15 - protein, 33 aa, synthetic construct
  MOD_RES 1: note = Type (i) lipidation
KCSTLSTCVLGKLSQELHKLQTYPRTNTGSGTP

SEQ ID NO: 16 - protein, 33 aa, synthetic construct
  MOD_RES 1: note = Type (i) lipidation
KCSNLSTCVLGKLSQELHKLQTYPRTPTGSGTP

SEQ ID NO: 17 - protein, 33 aa, synthetic construct
  MOD_RES 1: note = Type (i) lipidation
KCSNLSTCVLQKLSQELHKLQTYPRTNTGSGTP

SEQ ID NO: 18 - protein, 33 aa, synthetic construct
  MOD_RES 1: note = Type (i) lipidation
KCSNLSTCVLGKLSQELHKLQTYPRTNTGSNTP

SEQ ID NO: 19 - protein, 32 aa, synthetic construct
  MOD_RES 32: note = AMIDATION
  MOD_RES 1: note = Type (i) lipidation
KCNTATCVLGKLSQELHKLQTYPRTNTGSGTP

SEQ ID NO: 20 - protein, 32 aa, synthetic construct
  MOD_RES 32: note = AMIDATION
  MOD_RES 1: note = Type (i) lipidation
KCNTATCATQRLANFLHKLQTYPRTNTGSGTP

SEQ ID NO: 21 - protein, 32 aa, synthetic construct
  MOD_RES 32: note = AMIDATION
  MOD_RES 1: note = Type (i) lipidation
KCNTATCATQRLANELHKLQTYPRTNTGSGTP

SEQ ID NO: 22 - protein, 32 aa, synthetic construct
  MOD_RES 32: note = AMIDATION
  MOD_RES 1: note = Type (i) lipidation
KCNTATCVLGKLSQELHKLQTYPRTNTGSGTY

SEQ ID NO: 23 - protein, 33 aa, synthetic construct
  MOD_RES 33: note = AMIDATION
  MOD_RES 1: note = Type (i) lipidation
SKCNTATCVLGKLSQELHKLQTYPRTNTGSGTY

SEQ ID NO: 24 - protein, 33 aa, synthetic construct
  MOD_RES 33: note = AMIDATION
  MOD_RES 1: note = Type (i) lipidation
KKCNTATCVLGKLSQELHKLQTYPRTNTGSGTY

SEQ ID NO: 25 - protein, 33 aa, synthetic construct
  MOD_RES 33: note = AMIDATION
  MOD_RES 1: note = Type (i) lipidation
SKCNTATCVLGKLSQELHKLQTYPRTKTGSGTY

SEQ ID NO: 26 - protein, 33 aa, synthetic construct
  MOD_RES 33: note = AMIDATION
  MOD_RES 1: note = Type (i) lipidation
SKCNTATCVLGRLAQELHKLQTYPRTNTGSGTY

SEQ ID NO: 27 - protein, 33 aa, synthetic construct
  MOD_RES 33: note = AMIDATION
  MOD_RES 1: note = Type (i) lipidation
SKCNTATCVLGRLAQFLHKLQTYPRTNTGSGTY

SEQ ID NO: 28 - protein, 33 aa, synthetic construct
  MOD_RES 33: note = AMIDATION
  MOD_RES 1: note = Type (i) lipidation
SKCNTATCATQKLSQELHKLQTYPRTNTGSGTY

SEQ ID NO: 29 - protein, 33 aa, synthetic construct
  MOD_RES 33: note = AMIDATION
  MOD_RES 1: note = Type (i) lipidation
SKCNTATCATQRLAQFLHKLQTYPRTNTGSGTY

SEQ ID NO: 30 - protein, 33 aa, synthetic construct
  MOD_RES 33: note = AMIDATION
  MOD_RES 1: note = Type (i) lipidation
SKCNTATCVLGKLSQELHKLQTYPRTNVGSNTY

SEQ ID NO: 31 - protein, 32 aa, synthetic construct
  MOD_RES 32: note = AMIDATION
  MOD_RES 1: note = Type (i) lipidation
KCNTATCATQRLANELHKLQTYPRTNTGSGTF

SEQ ID NO: 32 - protein, 32 aa, synthetic construct
  MOD_RES 32: note = AMIDATION
  MOD_RES 1: note = Type (i) lipidation
KCNTATCATQRLANELHKLQTYPRTNTGSGTY

SEQ ID NO: 33 - protein, 33 aa, synthetic construct
  MOD_RES 33: note = AMIDATION
  MOD_RES 1: note = Type (i) lipidation
KKCNTATCATQRLANELHKLQTYPRTNTGSGTP

SEQ ID NO: 34 - protein, 32 aa, synthetic construct
  MOD_RES 32: note = AMIDATION
  MOD_RES 1: note = Type (i) lipidation
KCNTATCATQRLANELHKLQTYPRTPTGSGTP

SEQ ID NO: 35 - protein, 32 aa, synthetic construct
  MOD_RES 32: note = AMIDATION
  MOD_RES 1: note = Type (i) lipidation
KCNTATCATQRLANELHKLQTYPRTNTGSNTY

SEQ ID NO: 36 - protein, 32 aa, synthetic construct
  MOD_RES 32: note = AMIDATION
  MOD_RES 1: note = Type (i) lipidation
KCNTATCATQRLANELHKLQTYPRTNVGSNTY

SEQ ID NO: 37 - protein, 32 aa, synthetic construct
  MOD_RES 32: note = AMIDATION
  MOD_RES 1: note = Type (i) lipidation
KCNTATCATQRLANELHKLQTYPRTNVGSNTP

SEQ ID NO: 38 - protein, 32 aa, synthetic construct
  MOD_RES 32: note = AMIDATION
  MOD_RES 1: note = Type (i) lipidation
KCNTATCATQRLANELHKLQTYPRTNTGSGTA

SEQ ID NO: 39 - protein, 32 aa, synthetic construct
  MOD_RES 32: note = AMIDATION
  MOD_RES 1: note = Type (i) lipidation
KCNTATCATQRLANELHKLQTYPRTNTGSGTS

SEQ ID NO: 40 - protein, 32 aa, synthetic construct
  MOD_RES 32: note = AMIDATION
  MOD_RES 1: note = Type (i) lipidation
KCNTATCATQRLANELHKLQTYPRTNTGSGTK

SEQ ID NO: 41 - protein, 32 aa, synthetic construct
  MOD_RES 32: note = AMIDATION
  MOD_RES 1: note = Type (i) lipidation
KCNTATCATQRLANELHKLQTYPRTNTGSGTG

SEQ ID NO: 42 - protein, 32 aa, synthetic construct
  MOD_RES 32: note = AMIDATION
  MOD_RES 1: note = Type (i) lipidation
KCNTATCATQRLANELHKLQTYPRTNTGSGTH

SEQ ID NO: 43 - protein, 32 aa, synthetic construct
  MOD_RES 32: note = AMIDATION
  MOD_RES 1: note = Type (i) lipidation
KCNTATCATQRLANELHKLQTYPRTNTGSGTR

SEQ ID NO: 44 - protein, 33 aa, synthetic construct
  MOD_RES 33: note = AMIDATION
  MOD_RES 1: note = Type (ii) lipidation
EKCNTATCATQRLANELHKLQTYPRTNTGSGTP

SEQ ID NO: 45 - protein, 32 aa, synthetic construct
  MOD_RES 32: note = AMIDATION
  MOD_RES 1: note = Type (i) lipidation
KCSTATCATQRLANELHKLQTYPRTNTGSGTP

SEQ ID NO: 46 - protein, 32 aa, synthetic construct
  MOD_RES 32: note = AMIDATION
  MOD_RES 1: note = Type (i) lipidation
KCNTATCATQRLAQELHKLQTYPRTNTGSGTP

SEQ ID NO: 47 - protein, 32 aa, synthetic construct
  MOD_RES 32: note = AMIDATION
  MOD_RES 1: note = Type (i) lipidation
KCNTATCATQRLADELHKLQTYPRTNTGSGTP

SEQ ID NO: 48 - protein, 32 aa, synthetic construct
  MOD_RES 32: note = AMIDATION
  MOD_RES 1: note = Type (i) lipidation
KCNTATCATQRLANELHKLQTYPRTQTGSGTP

SEQ ID NO: 49 - protein, 32 aa, synthetic construct
  MOD_RES 32: note = AMIDATION
  MOD_RES 1: note = Type (i) lipidation
KCKTATCATQRLANELHKLQTYPRTNTGSGTP

SEQ ID NO: 50 - protein, 32 aa, synthetic construct
  MOD_RES 32: note = AMIDATION
  MOD_RES 1: note = Type (i) lipidation
KCKTATCATQRLANELHKLQTYPRTNTGSGTA

SEQ ID NO: 51 - protein, 33 aa, synthetic construct
  MOD_RES 33: note = AMIDATION
  MOD_RES 1: note = Type (i) lipidation
KKCNTATCATQRLANELHKLQTYPRTNTGSGTA

SEQ ID NO: 52 - protein, 34 aa, synthetic construct
  MOD_RES 34: note = AMIDATION
  MOD_RES 1: note = Type (ii) lipidation
EEKCKTATCATQRLANELHKLQTYPRTNTGSGTA

SEQ ID NO: 53 - protein, 33 aa, synthetic construct
  MOD_RES 33: note = AMIDATION
  MOD_RES 1: note = Type (ii) lipidation
EKCNTATCATQRLANELHKLQTYPRTNTGSGTA

SEQ ID NO: 54 - protein, 33 aa, synthetic construct
  MOD_RES 33: note = AMIDATION
  MOD_RES 1: note = Type (ii) lipidation
EKCKTATCATQRLANELHKLQTYPRTNTGSGTA

SEQ ID NO: 55 - protein, 33 aa, synthetic construct
  MOD_RES 33: note = AMIDATION
  MOD_RES 1: note = Type (ii) lipidation
EKCKTATCATQRLANELHKLQTYPRTNTGSGTP

SEQ ID NO: 56 - protein, 33 aa, synthetic construct; identical to SEQ ID NO: 33
  MOD_RES 33: note = AMIDATION
  MOD_RES 1: note = Type (i) lipidation

SEQ ID NO: 57 - protein, 32 aa, synthetic construct
  MOD_RES 32: note = AMIDATION
  MOD_RES 1: note = Type (i) lipidation
KCNTATCATQRLANELHKLQTYPRTNTGSNTA

SEQ ID NO: 58 - protein, 32 aa, synthetic construct
  MOD_RES 32: note = AMIDATION
  MOD_RES 1: note = Type (i) lipidation
KCNTATCATQRLANELHKLQTYPRTKTGSGTA

SEQ ID NO: 59 - protein, 32 aa, synthetic construct
  MOD_RES 32: note = AMIDATION
  MOD_RES 1: note = Type (i) lipidation
KCNTATCATQRLANELHKLQTYPRTPTGSGTA

SEQ ID NO: 60 - protein, 32 aa, synthetic construct
  MOD_RES 32: note = AMIDATION
  MOD_RES 1: note = Type (i) lipidation
KCKTATCATQRLANELHKLQTYPRTPTGSGTA

SEQ ID NO: 61 - protein, 32 aa, synthetic construct
  MOD_RES 32: note = AMIDATION
  MOD_RES 1: note = Type (i) lipidation
KCNTATCVLGRLAQELHKLQTYPRTNTGSGTY

SEQ ID NO: 62 - protein, 32 aa, synthetic construct
  MOD_RES 32: note = AMIDATION
  MOD_RES 1: note = Type (i) lipidation
KCNTATCVLGRLAQELHKLQTYPRTNTGSGTA

SEQ ID NO: 63 - protein, 32 aa, synthetic construct
  MOD_RES 32: note = AMIDATION
  MOD_RES 1: note = Type (i) lipidation
KCNTATCVLGRLAQELHKLQTYPRTNTGSGTP

SEQ ID NO: 64 - protein, 33 aa, synthetic construct
  MOD_RES 33: note = AMIDATION
  MOD_RES 1: note = Type (i) lipidation
KKCNTATCATQRLANELHKLQTYPRTPTGSGTP

SEQ ID NO: 65 - protein, 33 aa, synthetic construct
  MOD_RES 33: note = AMIDATION
  MOD_RES 1: note = Type (ii) lipidation
EKCNTATCVLGRLAQELHKLQTYPRTNTGSGTA

SEQ ID NO: 66 - protein, 32 aa, synthetic construct
  MOD_RES 32: note = AMIDATION
  MOD_RES 1: note = Type (i) lipidation
KCNTATCVLGRLAQELHKLQTYPRTNTGSNTA

SEQ ID NO: 67 - protein, 32 aa, synthetic construct
  MOD_RES 32: note = AMIDATION
  MOD_RES 1: note = Type (i) lipidation
KCNTATCVLGRLAQELHKLQTYPRTNVGSGTA

SEQ ID NO: 68 - protein, 32 aa, synthetic construct
  MOD_RES 32: note = AMIDATION
  MOD_RES 1: note = Type (i) lipidation
KCNTATCVLGRLAQELHKLQTYPRTNVGSNTA

SEQ ID NO: 69 - protein, 32 aa, synthetic construct
  MOD_RES 32: note = AMIDATION
  MOD_RES 1: note = Type (i) lipidation
KCNTATCVLGRLANELHKLQTYPRTNTGSGTP

SEQ ID NO: 70 - protein, 32 aa, synthetic construct
  MOD_RES 32: note = AMIDATION
  MOD_RES 1: note = Type (i) lipidation
KCNTATCATQRLANELHKLQTYPRTNTGSGTL

SEQ ID NO: 71 - protein, 32 aa, synthetic construct
  MOD_RES 32: note = AMIDATION
  MOD_RES 1: note = Type (i) lipidation
KCNTATCATQRLANELHKLQTYPRTNTGSGTV

SEQ ID NO: 72 - protein, 32 aa, synthetic construct
  MOD_RES 32: note = AMIDATION
  MOD_RES 1: note = Type (i) lipidation
KCNTATCATQRLANELHKLQTYPRTNTGSGTI

SEQ ID NO: 73 - protein, 32 aa, synthetic construct
  MOD_RES 32: note = AMIDATION
  MOD_RES 1: note = Type (i) lipidation
KCNTATCATQRLANELHKLQTYPRTNTGSGTQ

SEQ ID NO: 74 - protein, 32 aa, synthetic construct
  MOD_RES 32: note = AMIDATION
  MOD_RES 1: note = Type (i) lipidation
KCNTATCATQRLANELHKLQTYPRTNTGSGTE

SEQ ID NO: 75 - protein, 32 aa, synthetic construct
  MOD_RES 32: note = AMIDATION
  MOD_RES 1: note = Type (i) lipidation
KCNTATCATQRLANELHKLQTYPRTNTGSDTA

SEQ ID NO: 76 - protein, 32 aa, synthetic construct
  MOD_RES 32: note = AMIDATION
  MOD_RES 1: note = Type (i) lipidation
KCNTATCATQRLANELHRLQTYPRTNTGSGTA

SEQ ID NO: 77 - protein, 32 aa, synthetic construct; identical to SEQ ID NO: 36
  MOD_RES 32: note = AMIDATION
  MOD_RES 1: note = Type (i) lipidation

SEQ ID NO: 78 - protein, 32 aa, synthetic construct
  MOD_RES 32: note = AMIDATION
  MOD_RES 1: note = Type (i) lipidation
KCNTATCATQRLANELHKLQTYPRTNVGSNTA

SEQ ID NO: 79 - protein, 32 aa, synthetic construct; identical to SEQ ID NO: 37
  MOD_RES 32: note = AMIDATION
  MOD_RES 1: note = Type (i) lipidation

SEQ ID NO: 80 - protein, 32 aa, synthetic construct
  MOD_RES 32: note = AMIDATION
  MOD_RES 1: note = Type (i) lipidation
KCNTATCATQRLANELHKLQTYPRTNVGSDTP

SEQ ID NO: 81 - protein, 32 aa, synthetic construct
  MOD_RES 32: note = AMIDATION
  MOD_RES 1: note = Type (i) lipidation
KCNTATCATQRLANELHKLQTYPRTNVGSKTP

SEQ ID NO: 82 - protein, 32 aa, synthetic construct
  MOD_RES 32: note = AMIDATION
  MOD_RES 1: note = Type (i) lipidation
KCNTATCATQRLANELHKLQTYPRTDVGSNTP

SEQ ID NO: 83 - protein, 32 aa, synthetic construct
  MOD_RES 32: note = AMIDATION
  MOD_RES 1: note = Type (i) lipidation
KCNTATCATQRLANELHKLQTYPRTKVGSNTP

SEQ ID NO: 84 - protein, 33 aa, synthetic construct
  MOD_RES 33: note = AMIDATION
  MOD_RES 1: note = Type (i) lipidation
KCSNLSTCMLGRLSQELHRLQTFPKTDVGANAP

SEQ ID NO: 85 - protein, 33 aa, synthetic construct
  MOD_RES 33: note = AMIDATION
  MOD_RES 1: note = Type (i) lipidation
KCSNLSTCMLGRLSQDLHRLQTYPKTDVGANAP

SEQ ID NO: 86 - protein, 33 aa, synthetic construct
  MOD_RES 33: note = AMIDATION
  MOD_RES 1: note = Type (i) lipidation
KCSNLSTCVLGKLSQELHKLQTYPRTDVGANAP

SEQ ID NO: 87 - protein, 33 aa, synthetic construct
  MOD_RES 33: note = AMIDATION
  MOD_RES 1: note = Type (i) lipidation
SKCNTATCATQRLANELHKLQTYPRTNTGSGTP

SEQ ID NO: 88 - protein, 33 aa, synthetic construct
  MOD_RES 33: note = AMIDATION
  MOD_RES 1: note = Type (i) lipidation
SKCNTATCATQRLANELHKLQTYPRTNTGSGTA

SEQ ID NO: 89 - protein, 33 aa, synthetic construct
  MOD_RES 33: note = AMIDATION
  MOD_RES 1: note = Type (i) lipidation
SKCNTATCATQRLANELHKLQTYPRTNTGSNTA

SEQ ID NO: 90 - protein, 33 aa, synthetic construct
  MOD_RES 33: note = AMIDATION
  MOD_RES 1: note = Type (ii) lipidation
EKCNTATCATQRLANELHKLQTYPRTNVGSNTP

SEQ ID NO: 91 - protein, 33 aa, synthetic construct
  MOD_RES 33: note = AMIDATION
  MOD_RES 1: note = Type (ii) lipidation
EKCNTATCATQRLANELHKLQTYPRTQTGSGTP

SEQ ID NO: 92 - protein, 33 aa, synthetic construct
  MOD_RES 33: note = AMIDATION
  MOD_RES 1: note = Type (ii) lipidation
EKCNTATCATQRLANELHKLQTYPRTQVGSNTP

SEQ ID NO: 93 - protein, 33 aa, synthetic construct
  MOD_RES 33: note = AMIDATION
  MOD_RES 1: note = Type (ii) lipidation
EKCNTATCATQRLAQELHKLQTYPRTNVGSNTP

SEQ ID NO: 94 - protein, 33 aa, synthetic construct
  MOD_RES 33: note = AMIDATION
  MOD_RES 1: note = Type (ii) lipidation
EKCNTATCATQRLAQELHKLQTYPRTQVGSNTP

SEQ ID NO: 95 - protein, 33 aa, synthetic construct
  MOD_RES 33: note = AMIDATION
  MOD_RES 1: note = Type (ii) lipidation
EKCNTATCATQRLAKELHKLQTYPRTQVGSNTP

SEQ ID NO: 96 - protein, 34 aa, synthetic construct
  MOD_RES 34: note = AMIDATION
  MOD_RES 1: note = Type (ii) lipidation
EKKCNTATCATQRLANELHKLQTYPRTNVGSNTP

SEQ ID NO: 97 - protein, 33 aa, synthetic construct
  MOD_RES 33: note = AMIDATION
  MOD_RES 1: note = Type (ii) lipidation
EKCNTATCATQRLADELHKLQTYPRTQVGSNTP

SEQ ID NO: 98 - protein, 33 aa, synthetic construct
  MOD_RES 33: note = AMIDATION
  MOD_RES 1: note = Type (ii) lipidation
EKCNTATCATQRLADELHKLQTYPRTPVGSNTP

SEQ ID NO: 99 - protein, 33 aa, synthetic construct
  MOD_RES 33: note = AMIDATION
  MOD_RES 1: note = Type (ii) lipidation
EKCNTATCATQRLADELHKLQTYPRTPTGSNTP

SEQ ID NO: 100 - protein, 33 aa, synthetic construct
  MOD_RES 33: note = AMIDATION
  MOD_RES 1: note = Type (ii) lipidation
EKCSTATCATQRLADELHKLQTYPRTPTGSNTP

SEQ ID NO: 101 - protein, 33 aa, synthetic construct
  MOD_RES 33: note = AMIDATION
  MOD_RES 1: note = Type (ii) lipidation
EKCSTATCATQRLADELHKLQTYPRTQVGSNTP

SEQ ID NO: 102 - protein, 32 aa, synthetic construct
  MOD_RES 32: note = AMIDATION
  MOD_RES 1: note = Type (i) lipidation
KCNTATCATQRLADELHKLQTYPRTPTGSGTP

SEQ ID NO: 103 - protein, 32 aa, synthetic construct
  MOD_RES 32: note = AMIDATION
  MOD_RES 1: note = Type (i) lipidation
KCNTATCATQRLADELHKLQTYPRTPVGSNTP

SEQ ID NO: 104 - protein, 33 aa, synthetic construct
  MOD_RES 33: note = AMIDATION
  MOD_RES 1: note = Type (i) lipidation
KKCNTATCATQRLADELHKLQTYPRTPVGSNTP

SEQ ID NO: 105 - protein, 32 aa, synthetic construct
  MOD_RES 32: note = AMIDATION
  MOD_RES 1: note = Type (i) lipidation
KCNTATCATQRLANELHKLQTYPRTKTGSGTP

SEQ ID NO: 106 - protein, 32 aa, synthetic construct
  MOD_RES 32: note = AMIDATION
  MOD_RES 1: note = Type (i) lipidation
KCNTATCATQRLANELHKLKTYPRTNTGSGTP

SEQ ID NO: 107 - protein, 33 aa, synthetic construct
  MOD_RES 33: note = AMIDATION
  MOD_RES 1: note = Type (ii) lipidation
EKCKTATCATQRLADELHKLQTYPRTPVGSNTP

SEQ ID NO: 108 - protein, 32 aa, synthetic construct
  MOD_RES 32: note = AMIDATION
  MOD_RES 1: note = Type (i) lipidation
KCKTATCATQRLADELHKLQTYPRTPVGSNTP

SEQ ID NO: 109 - protein, 32 aa, synthetic construct
  MOD_RES 32: note = AMIDATION
  MOD_RES 1: note = Type (i) lipidation
KCKTATCATQRLADELHKLQTYPRTPVGSKTP

SEQ ID NO: 110 - protein, 32 aa, synthetic construct
  MOD_RES 32: note = AMIDATION
  MOD_RES 1: note = Type (i) lipidation
KCKTATCATQRLANELHKLQTYPRTPVGSKTP

SEQ ID NO: 111 - protein, 32 aa, synthetic construct
  MOD_RES 32: note = AMIDATION
  MOD_RES 1: note = Type (i) lipidation
KCKTATCATQRLADELHKLQTYPRTNVGSKTP

SEQ ID NO: 112 - protein, 33 aa, synthetic construct
  MOD_RES 33: note = AMIDATION
  MOD_RES 1: note = Type (i) lipidation
SKCKTATCATQRLANELHKLQTYPRTPVGSDTP

SEQ ID NO: 113 - protein, 33 aa, synthetic construct
  MOD_RES 33: note = AMIDATION
  MOD_RES 1: note = Type (i) lipidation
SKCKTATCATQRLADELHKLQTYPRTPVGSDTP

SEQ ID NO: 114 - protein, 33 aa, synthetic construct
  MOD_RES 33: note = AMIDATION
  MOD_RES 1: note = Type (i) lipidation
SKCKTATCATQRLAEELHKLQTYPRTPVGSDTP

SEQ ID NO: 115 - protein, 33 aa, synthetic construct
  MOD_RES 33: note = AMIDATION
  MOD_RES 1: note = Type (i) lipidation
SKCKTATCATQRLAQELHKLQTYPRTPVGSDTP

SEQ ID NO: 116 - protein, 33 aa, synthetic construct
  MOD_RES 33: note = AMIDATION
  MOD_RES 1: note = Type (i) lipidation
SKCKTATCATQRLAKELHKLQTYPRTPVGSDTP

SEQ ID NO: 117 - protein, 33 aa, synthetic construct
  MOD_RES 33: note = AMIDATION
  MOD_RES 1: note = Type (i) lipidation
KKCKTATCATQRLANELHKLQTYPRTPVGSDTP

SEQ ID NO: 118 - protein, 33 aa, synthetic construct
  MOD_RES 33: note = AMIDATION
  MOD_RES 1: note = Type (i) lipidation
KKCKTATCATQRLADELHKLQTYPRTPVGSDTP

SEQ ID NO: 119 - protein, 33 aa, synthetic construct
  MOD_RES 33: note = AMIDATION
  MOD_RES 1: note = Type (i) lipidation
KKCKTATCATQRLAEELHKLQTYPRTPVGSDTP

SEQ ID NO: 120 - protein, 33 aa, synthetic construct
  MOD_RES 33: note = AMIDATION
  MOD_RES 1: note = Type (i) lipidation
KKCKTATCATQRLAQELHKLQTYPRTPVGSDTP

SEQ ID NO: 121 - protein, 33 aa, synthetic construct
  MOD_RES 33: note = AMIDATION
  MOD_RES 1: note = Type (i) lipidation
KKCKTATCATQRLAKELHKLQTYPRTPVGSDTP

SEQ ID NO: 122 - protein, 33 aa, synthetic construct
  MOD_RES 33: note = AMIDATION
  MOD_RES 1: note = Type (i) lipidation
KKCKTATCATQRLANELHKLQTYPRTPVGSKTP

SEQ ID NO: 123 - protein, 32 aa, synthetic construct
  MOD_RES 32: note = AMIDATION
  MOD_RES 1: note = Type (i) lipidation
KCNTATCATQRLAQELHKLQTYPRTNVGSKTP

SEQ ID NO: 124 - protein, 32 aa, synthetic construct
  MOD_RES 32: note = AMIDATION
  MOD_RES 1: note = Type (i) lipidation
KCNTATCATQRLAQELHKLQTYPRTPVGSKTP

SEQ ID NO: 125 - protein, 32 aa, synthetic construct
  MOD_RES 32: note = AMIDATION
  MOD_RES 1: note = Type (i) lipidation
KCNTATCATQRLAQELHKLQTYPRTKVGSKTP

SEQ ID NO: 126 - protein, 32 aa, synthetic construct
  MOD_RES 32: note = AMIDATION
  MOD_RES 1: note = Type (i) lipidation
KCKTATCATQRLAQELHKLQTYPRTNVGSKTP

SEQ ID NO: 127 - protein, 32 aa, synthetic construct
  MOD_RES 32: note = AMIDATION
  MOD_RES 1: note = Type (i) lipidation
KCKTATCATQRLAQELHKLQTYPRTKVGSKTP

SEQ ID NO: 128 - protein, 32 aa, synthetic construct
  MOD_RES 32: note = AMIDATION
  MOD_RES 1: note = Type (i) lipidation
KCNTATCATQRLADELHKLQTYPRTNVGSKTP

SEQ ID NO: 129 - protein, 32 aa, synthetic construct
  MOD_RES 32: note = AMIDATION
  MOD_RES 1: note = Type (i) lipidation
KCNTATCATQRLAEELHKLQTYPRTNVGSKTP

SEQ ID NO: 130 - protein, 32 aa, synthetic construct
  MOD_RES 32: note = AMIDATION
  MOD_RES 1: note = Type (i) lipidation
KCSTATCATQRLADELHKLQTYPRTNVGSNTP

SEQ ID NO: 131 - protein, 32 aa, synthetic construct
  MOD_RES 32: note = AMIDATION
  MOD_RES 1: note = Type (i) lipidation
KCSTATCATQRLAEELHKLQTYPRTNVGSNTP

SEQ ID NO: 132 - protein, 32 aa, synthetic construct
  MOD_RES 32: note = AMIDATION
  MOD_RES 1: note = Type (i) lipidation
KCSTATCATQRLADELHKLQTYPRTNTGSGTP

SEQ ID NO: 133 - protein, 32 aa, synthetic construct
  MOD_RES 32: note = AMIDATION
  MOD_RES 1: note = Type (i) lipidation
KCSTATCATQRLAEELHKLQTYPRTNTGSGTP

SEQ ID NO: 134 - protein, 32 aa, synthetic construct
  MOD_RES 32: note = AMIDATION
  MOD_RES 1: note = Type (i) lipidation
KCSTATCATQRLAEELHKLQTYPRTKTGSGTP

SEQ ID NO: 135 - protein, 33 aa, synthetic construct
  MOD_RES 33: note = AMIDATION
  MOD_RES 1: note = Type (i) lipidation
KKCNTATCATQRLADELHKLKTYPRTPVGSNTP

SEQ ID NO: 136 - protein, 33 aa, synthetic construct
  MOD_RES 33: note = AMIDATION
  MOD_RES 1: note = Type (i) lipidation
KKCNTATCATQRLAEELHKLKTYPRTPVGSNTP

SEQ ID NO: 137 - protein, 33 aa, synthetic construct
  MOD_RES 33: note = AMIDATION
  MOD_RES 1: note = Type (i) lipidation
KKCSTATCATQRLADELHKLKTYPRTPVGSNTP

SEQ ID NO: 138 - protein, 33 aa, synthetic construct
  MOD_RES 33: note = AMIDATION
  MOD_RES 1: note = Type (i) lipidation
KKCNTATCATQRLADELHKLQTYPRTPVGSKTP

SEQ ID NO: 139 - protein, 33 aa, synthetic construct
  MOD_RES 33: note = AMIDATION
  MOD_RES 1: note = Type (i) lipidation
KKCSTATCATQRLADELHKLKTYPRTPVGSKTP

SEQ ID NO: 140 - protein, 33 aa, synthetic construct
  MOD_RES 33: note = AMIDATION
  MOD_RES 1: note = Type (i) lipidation
KKCNTATCATQRLADELHKLKTYPRTPVGSKTP

SEQ ID NO: 141 - protein, 33 aa, synthetic construct
  MOD_RES 33: note = AMIDATION
  MOD_RES 1: note = Type (i) lipidation
KKCSTATCATQRLADELHKLQTYPRTPVGSKTP

SEQ ID NO: 142 - protein, 33 aa, synthetic construct
  MOD_RES 33: note = AMIDATION
  MOD_RES 1: note = Type (i) lipidation
KKCNTATCATQRLAEELHKLQTYPRTPVGSNTP

SEQ ID NO: 143 - protein, 33 aa, synthetic construct; identical to SEQ ID NO: 140
  MOD_RES 33: note = AMIDATION
  MOD_RES 1: note = Type (i) lipidation

SEQ ID NO: 144 - protein, 33 aa, synthetic construct
  MOD_RES 33: note = AMIDATION
  MOD_RES 1: note = Type (i) lipidation
KKCSTATCATQRLAEELHKLQTYPRTPVGSNTP

SEQ ID NO: 145 - protein, 33 aa, synthetic construct
  MOD_RES 33: note = AMIDATION
  MOD_RES 1: note = Type (i) lipidation
KKCNTATCATQRLAQELHKLQTYPRTPVGSNTP

SEQ ID NO: 146 - protein, 33 aa, synthetic construct
  MOD_RES 33: note = AMIDATION
  MOD_RES 1: note = Type (i) lipidation
KKCSTATCATQRLAEELHKLQTYPRTPVGSKTP

(19) United States
(12) Patent Application Publication
(10) Pub. No.: US 2026/0152538 A1
(43) Pub. Date: Jun. 4, 2026

(54) AMYLIN ANALOGUES

(71) Applicant: IP2IPO Innovations Limited, London (GB)

(72) Inventor: Stephen Robert BLOOM, London (GB)

(21) Appl. No.: 19/132,752

(22) PCT Filed: Nov. 24, 2023
(86) PCT No.: PCT/GB2023/053067

§ 371 Date: May 23, 2025

(30) Foreign Application Priority Data
Nov. 24, 2022 (GB) 2217575.6

(51) Int. Cl. C07K 14/575; A61K 38/00; A61P 3/04

(52) U.S. Cl. CPC C07K 14/575; A61P 3/04; A61K 38/00

(57) ABSTRACT

A compound comprising a peptide chain which is an analogue of amylin, a terminal —NH₂ group bound to the C terminal of the peptide chain, and an alkylene or alkenylene chain attached at the other end of the peptide chain or a derivative of the compound; or a salt or solvate of the compound or of the derivative. Compounds, derivates and salts, and related compositions are suitable for the prevention or treatment of diabetes, obesity, heart disease, stroke and non-alcoholic fatty liver disease, improving insulin release in a subject, improving carbohydrate metabolism in a subject, improving the lipid profile of a subject, improving carbohydrate tolerance in a subject, reducing appetite, reducing food intake, and/or reducing calorie intake, and related purposes.

### FIELD

The present disclosure relates to compounds which are peptide hormone analogues, and which are useful in treating disorders such as diabetes and obesity.

### BACKGROUND

According to the National Health and Nutrition Examination Survey (NHANES, 2009-2010), 33.0% of adults in the United States aged 20 and over were overweight, 35.7% were obese, and 6.3% were severely obese. Since then obesity prevalence in the United States has risen further with 41.9% of US adults classified as obese in 2020 and 9.2% classified as severely obese (NHANES, 2020). In addition, a large percentage of children in the United States are overweight or obese.

The cause of obesity is complex and multi-factorial. Increasing evidence suggests that obesity is not a simple problem of self-control but is a complex disorder involving appetite regulation and energy metabolism. In addition, obesity is associated with a variety of conditions associated with increased morbidity and mortality in a population. Although the etiology of obesity is not definitively established, genetic, metabolic, biochemical, cultural and psychosocial factors are believed to contribute. In general, obesity has been described as a condition in which excess body fat puts an individual at a health risk.

There is strong evidence that obesity is associated with increased morbidity and mortality. Disease risk, such as cardiovascular disease risk and type 2 diabetes disease risk, increases independently with increased body mass index (BMI). Indeed, this risk has been quantified as a five percent increase in the risk of cardiac disease for females, and a seven percent increase in the risk of cardiac disease for males, for each point of a BMI greater than 24.9 (see Kenchaiah et al., N. Engl. J. Med. 347:305, 2002; Massie, N. Engl. J. Med. 347:358, 2002).

Diabetes is a chronic syndrome of impaired carbohydrate, protein, and fat metabolism owing to insufficient secretion of insulin or to target tissue insulin resistance. It occurs in two major forms: insulin-dependent diabetes mellitus (type 1 diabetes) and non-insulin dependent diabetes mellitus (type 2 diabetes). Diabetes type I, or insulin dependent diabetes mellitus (IDDM) is caused by the destruction of β cells, which results in insufficient levels of endogenous insulin. Diabetes type 2, or non-insulin dependent diabetes, results from a defect in both the body's sensitivity to insulin, and a relative deficiency in insulin production. According to the National Diabetes Statistics Report, 2014 around 28.9 million adults in the United States aged 20 and over have diabetes (2009-2012 National Health and Nutrition Examination Survey estimates applied to 2012 U.S. Census data). In adults 90 to 95% of the diabetes is type 2 diabetes.

There is substantial evidence that weight loss in obese persons reduces important disease risk factors. Even a small weight loss, such as 10% of the initial body weight in both overweight and obese adults has been associated with a decrease in risk factors such as hypertension, hyperlipidemia, and hyperglycemia. It has been shown that considerable weight loss can effectively cure type 2 diabetes (Lim et al, Diabetologia June 2011).

Although diet and exercise provide a simple method to decrease weight gain, overweight and obese individuals often cannot sufficiently control these factors to effectively lose weight. Pharmacotherapy is available; several weight loss drugs have been approved by the Food and Drug Administration that can be used as part of a comprehensive weight loss program. However, many of these drugs have serious adverse side effects. When less invasive methods have failed, and the patient is at high risk for obesity related morbidity or mortality, weight loss surgery is an option in carefully selected patients with clinically severe obesity. However, these treatments are high-risk, and suitable for use in only a limited number of patients. It is not only obese subjects who wish to lose weight. People with weight within the recommended range, for example in the upper part of the recommended range, may wish to reduce their weight, to bring it closer to their ideal weight. Thus, a need remains for agents that can be used to effect weight loss in overweight and obese subjects as well as subjects who are of normal weight.

A number of approaches to the development of agents useful in effecting weight loss have involved gastrointestinal peptide hormones and their analogues.

However, despite significant advances, the process of identifying substances useful as drugs remains a complex and, in many cases, unpredictable field. In order to be useful as therapeutic agents, compounds must possess a suitable range of properties. In addition to having good efficacy at the biological target of interest, compounds must have good in vivo pharmacokinetic properties, low toxicity and an acceptable side effect profile. For example, even with commercial agents such as liraglutide, side effects can include nausea and vomiting, and concerns have also been raised with regard to thyroid cancer and pancreatitis.

Thus, there remains a need for further compounds which are useful for the treatment of disorders and diseases such as diabetes and obesity. For example, it would be desirable to identify peptides having beneficial properties such as an improved activity profile, and/or which have reduced side effects. For example, it would be desirable for a peptide to be identified that increases energy expenditure in a subject, but does not significantly reduce food intake. If a compound decreases food intake less, then it is expected that the compound will have fewer side effects such as nausea. Alternatively, or additionally, it would be desirable for a peptide to be identified that has these and other biological effects for a sustained period. A compound that has a longer period of activity can be administered less frequently and at lower dose, which contributes to improved convenience for the subject, to fewer side effects and to lower cost.

Amylin (also called islet amyloid polypeptide, IAPP) is a 37 residue peptide hormone which is co-secreted with insulin from pancreatic beta cells. It is known to play a role in glycaemic regulation, slow gastric emptying and increase satiety. Its production may become deficient in subjects having type 2 diabetes in which there is loss of pancreatic beta cells.

Human amylin has amyloidogenic properties meaning that it has an ability to form insoluble precipitates and fibril both intracellularly and extracellularly. These amyloid fibrils and precipitates are known to be cytotoxic and have been implicated in both neurodegeneration and death of beta cells of the pancreas.

Amyloidogenic activity is present in human amylin and also amylin from at least some other primates. Amylin from other species, including mice and rats lacks residues implicated in amyloidogenic activity. Amylin Pharmaceuticals Inc. market the drug Pramlintide Acetate (Symlin™), which is the acetate salt of an analogue of human amylin in which residues 25, 26, 29 of human amylin are substituted in order to reduce (although not completely eliminate) amyloidogenic activity. Pramlintide Acetate was FDA approved in 2005 for use in diabetics who use insulin but who have difficulty in maintaining glycaemic control. It allows patients to use less insulin and results in reduced undershoots and overshoots of blood sugar. It is has the property of promoting satiety but has a low potency in reducing unhealthy weight and is not approved for use as a weight loss drug.

Novo Nordisk has a potential obesity management drug in clinical trials called Cagrilintide where it is co-administered with Semaglutide. Cagrilintide has a peptide sequence which is rather similar to that of Pramlintide. The sequence so all the peptides discussed are given below:

Pramlintide
(SEQ ID NO.: 1)
KCNTATCATQRLANFLVHSSNNFGPILPPTNVGSNTY-(NH2)
Cagrilintide
(SEQ ID NO.: 2)
KCNTATCATQRLAEFLRHSSNNFGPILPPTNVGSNTP-(NH2)
Human amylin
(SEQ ID NO.: 3)
KCNTATCATQRLANFLVHSSNNFGAILSSTNVGSNTY-(NH2)
Rat amylin
(SEQ ID. NO.: 4)
KCNTATCATQRLANFLVRSSNNLGPVLPPTNVGSNTY-(NH2)

Amylin, Cagrilintide and Pramlintide all amidated at their C-terminus. All of these peptides also have a —S—S— bridge between the two C residues. For an injectable weight loss drug to be acceptable to patients and cost-acceptable, it preferably has a post-injection half-life sufficient of at least several days and preferably a week or more so that it can be conveniently administered with an interval of a week or longer. All of the peptides shown above have a short life. The drug Cagrilintide (but not Amylin nor Pramlintide) is lipidated in order to increase its half-life to the extent that a once weekly injection regime may be within reach.

There remains a need for improved therapeutic weight loss agents. Despite significant advances, the process of identifying substances useful as drugs remains a complex and, in many cases, unpredictable field. In order to be useful as therapeutic agents, compounds must possess a suitable range of properties. In addition to having good efficacy at the biological target of interest, compounds must have good in vivo pharmacokinetic properties, low toxicity and an acceptable side effect profile. For example, even with commercial agents such as liraglutide, side effects can include nausea and vomiting, and concerns have also been raised with regard to thyroid cancer and pancreatitis.

Thus, there remains a need for further compounds which are useful for the treatment of disorders and diseases such as diabetes and obesity. For example, it would be desirable to identify peptides having beneficial properties such as an improved activity profile, and/or which have reduced side effects. For example, it would be desirable for a peptide to be identified that increases energy expenditure in a subject, but does not significantly reduce food intake. If a compound decreases food intake less, then it is expected that the compound will have fewer side effects such as nausea. Alternatively, or additionally, it would be desirable for a peptide to be identified that has these and other biological effects for a sustained period. A compound that has a longer period of activity can be administered less frequently and at lower dose, which contributes to improved convenience for the subject, to fewer side effects and to lower cost. The present invention is provides novel weight loss agents which are based on Amylin but which have advantages over Cagrilintide including greater potency, longer half-life and lower cost.

### SUMMARY OF THE INVENTION

In a first aspect of the invention there is provided a compound of formula:

A-B-C, wherein:

A is Q, Q-Ser, Q-Glu or Q-Lys, wherein Q is a group of the formula:

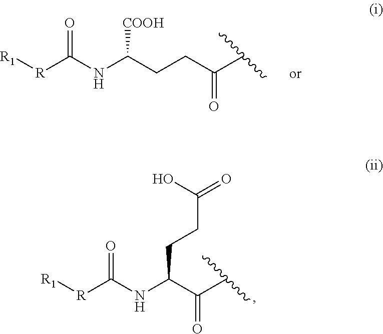

wherein R is a C₈-C₂₈ alkylene or alkenylene chain and R₁ is —CO₂H;
B is a peptide moiety of 32 or 33 amino acid residues, having a sequence of X-Y-Z. wherein:
X is Lys-Cys
Y is Xaa1-Xaa2-Xaa3-Xaa4-Xaa5-Xaa6, wherein;
Xaa1 is Ser or absent
Xaa2 is Asn, Lys, Ser or Thr
Xaa3 is Thr or Leu
Xaa4 is Ala or Ser
Xaa5 is Thr
Xaa6 is Cys
Z is Xaa7-Xaa8-Xaa9-Xaa10-Xaa11-Xaa12-Xaa13-Xaa14-Xaa15-Xaa16-Xaa17-Xaa18-Xaa19-Xaa20-Xaa21-Xaa22-Xaa23-Xaa24-Xaa25-Xaa26-Xaa27-Xaa28-Xaa29-Xaa30-Xaa31 (SEQ ID. NO.: 6), wherein:
Xaa7 is Ala, Val or Met
Xaa8 is Thr or Leu
Xaa9 is Gln or Gly
Xaa10 is Arg or Lys
Xaa11 is Leu
Xaa12 is Ala or Ser
Xaa13 is any amino acid
Xaa14 is Glu, Asp or Phe
Xaa15 is Leu
Xaa16 is His
Xaa17 is Lys or Arg
Xaa18 is Leu
Xaa19 is Gln or Lys
Xaa20 is Thr
Xaa21 is Tyr or Phe
Xaa22 is Pro
Xaa23 is Arg or Lys
Xaa24 is Thr
Xaa25 is Gln, Asp, Pro, Lys or Asn
Xaa26 is Val or Thr
Xaa27 is Gly
Xaa28 is Ser or Ala
Xaa29 is Lys, Asn, Gly, or Asp
Xaa30 is Thr or Ala
Xaa31 is any amino acid
C is a terminal —NH₂ group bound to the C terminal of peptide moiety B
or a derivative of the compound; or a salt or solvate of the compound or of the derivative

Also provided herein is a composition comprising a compound, derivative, salt or solvate of the invention together with a pharmaceutically acceptable carrier and optionally a further therapeutic agent.

Also provided herein is a compound, derivative, salt or solvate of the invention, or a composition comprising such a compound, derivative, salt or solvate and a pharmaceutically acceptable carrier, for use as a medicament, e.g. for use in the prevention or treatment of diabetes, obesity, heart disease, stroke or non-alcoholic fatty liver disease, improving insulin release in a subject, improving carbohydrate metabolism in a subject, improving the lipid profile of a subject, reducing appetite, reducing food intake, reducing calorie intake, and/or improving carbohydrate tolerance in a subject.

Also provided herein is a method of treating or preventing a disease or disorder or other non-desired physiological state in a subject comprising administration of a therapeutically effective amount of a compound, derivative, salt or solvate of the invention, or of a composition comprising such a compound, derivative, salt or solvate and a pharmaceutically acceptable carrier, e.g. in a method of treating or preventing diabetes, obesity, heart disease, stroke or non-alcoholic fatty liver disease in a subject, improving insulin release in a subject, improving carbohydrate metabolism in a subject, improving the lipid profile of a subject, improving carbohydrate tolerance in a subject, reducing appetite, reducing food intake, and/or reducing calorie intake in a subject.

Also provided herein is a use of a compound, derivative, salt or solvate of the invention for the manufacture of a medicament for the prevention or treatment of diabetes, obesity, heart disease, stroke or non-alcoholic fatty liver disease, improving insulin release in a subject, improving carbohydrate metabolism in a subject, improving the lipid profile of a subject, improving carbohydrate tolerance in a subject, reducing appetite, reducing food intake and/or reducing calorie intake.

Also provided herein is a method of causing weight loss or preventing weight gain in a subject for cosmetic purposes comprising administration of an effective amount of a compound, derivative, salt or solvate of the invention.

### BRIEF DESCRIPTION OF THE DRAWINGS

FIG. 1 shows, in a table spread over multiple pages, the amino acid sequences of example compounds of the invention. The compounds are presented with the N-terminal residue at the left-hand side of the table. Amino acid sequence shown in FIG. 1 which are not part of compounds of the invention, such as prior art and naturally-occurring sequences are included solely for ease of comparison.

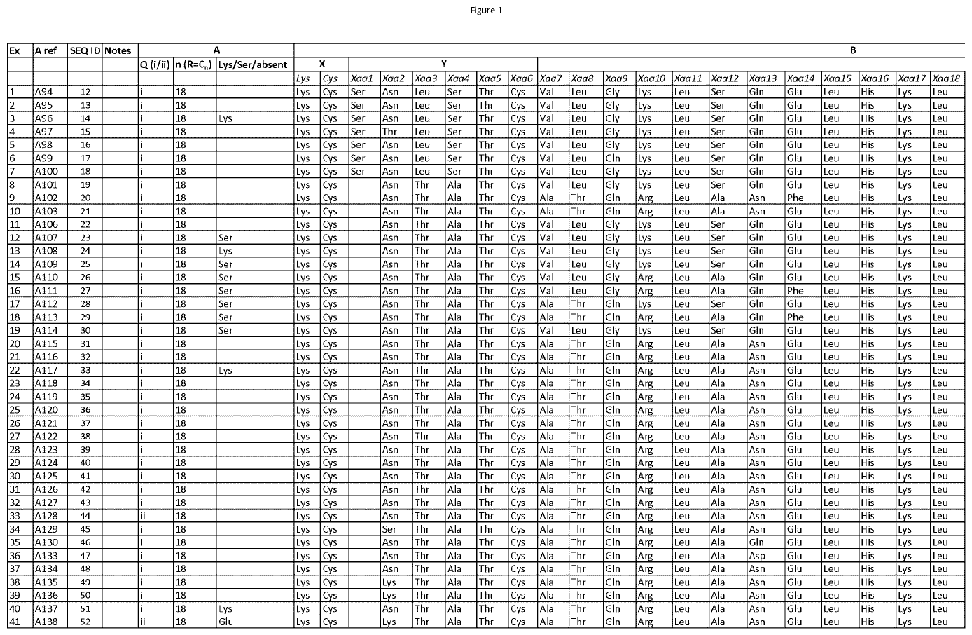

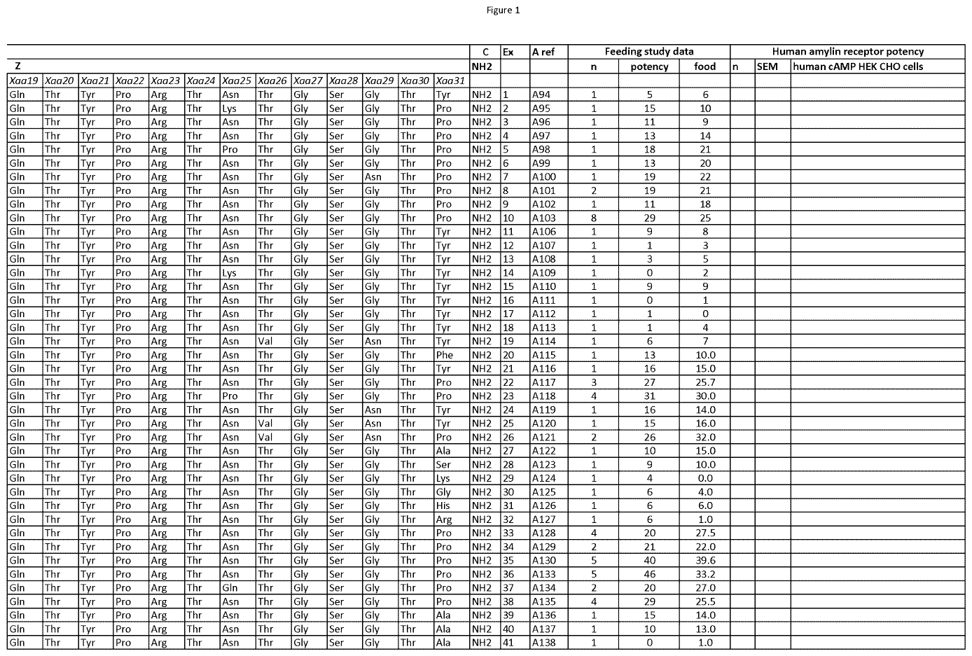

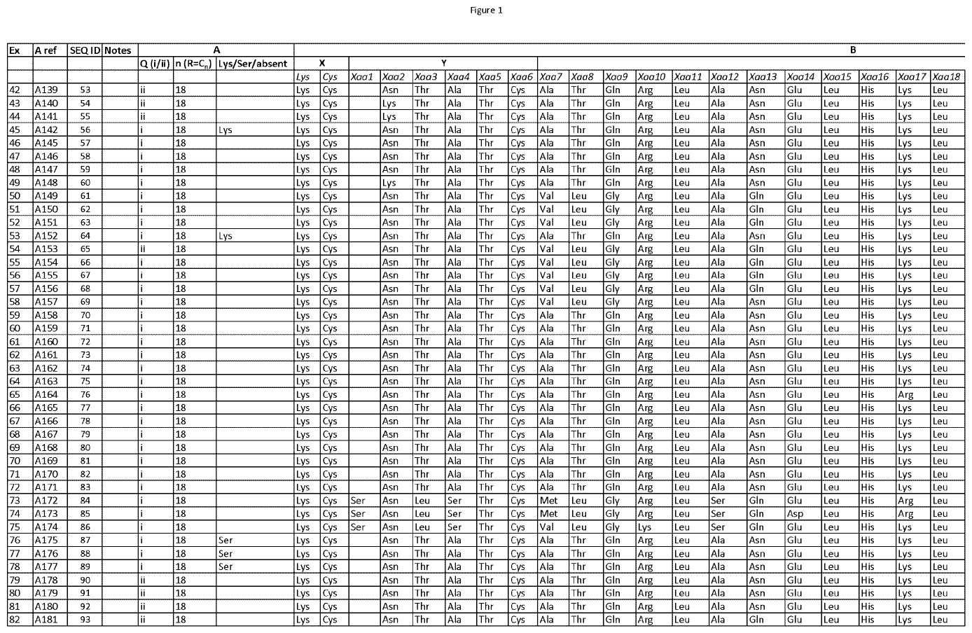

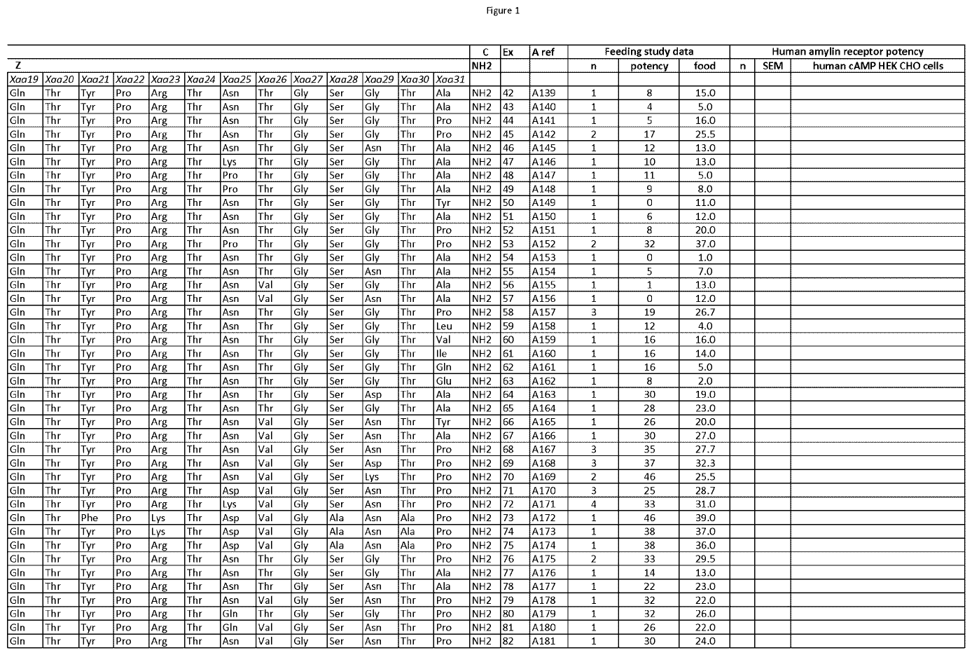

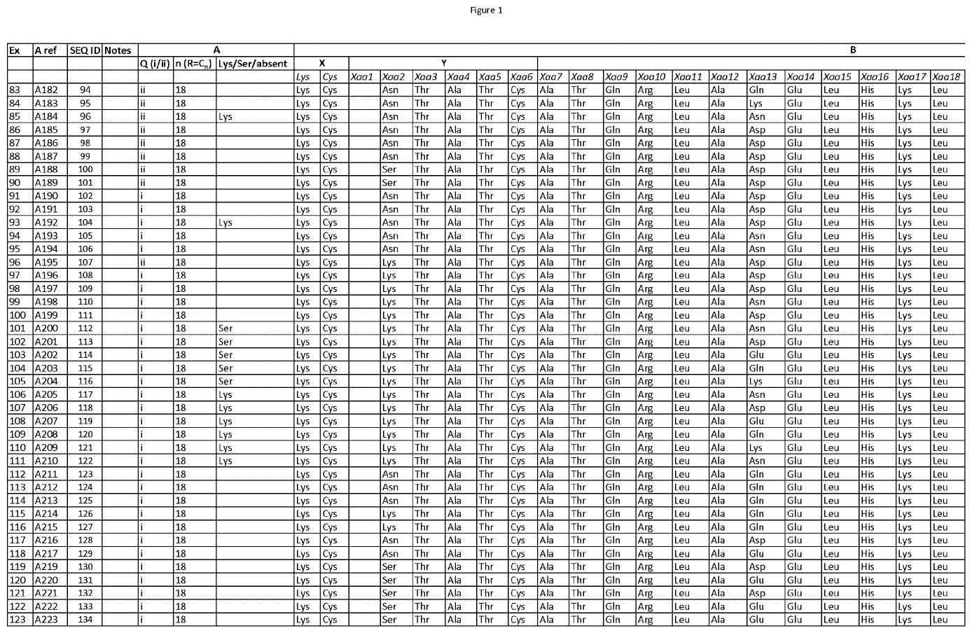

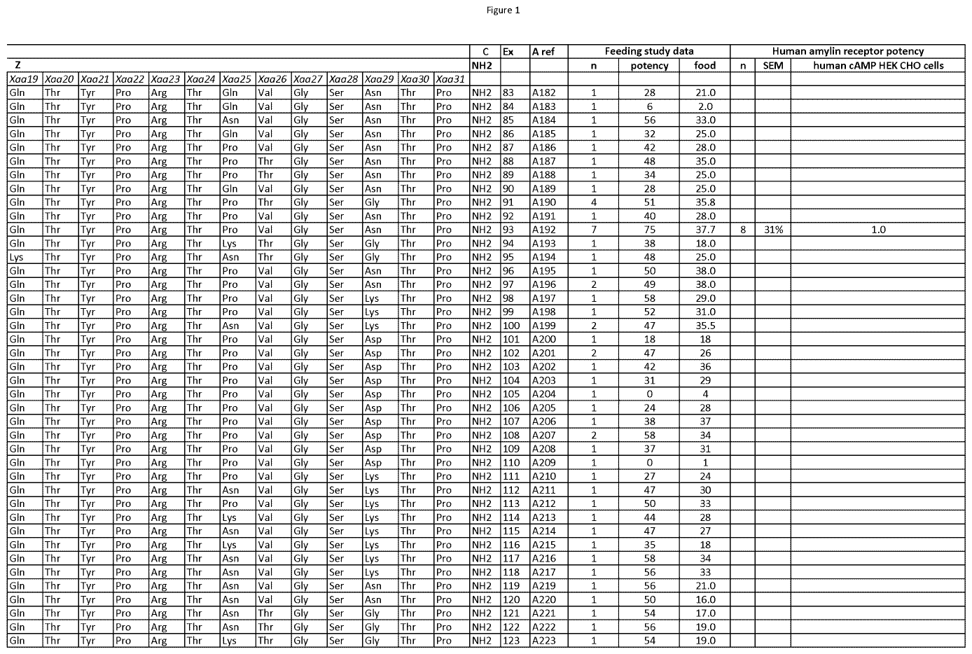

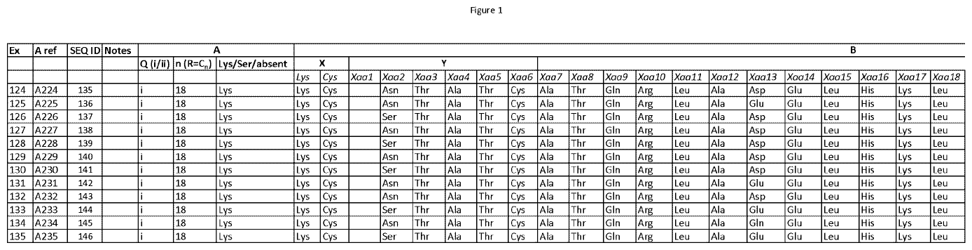

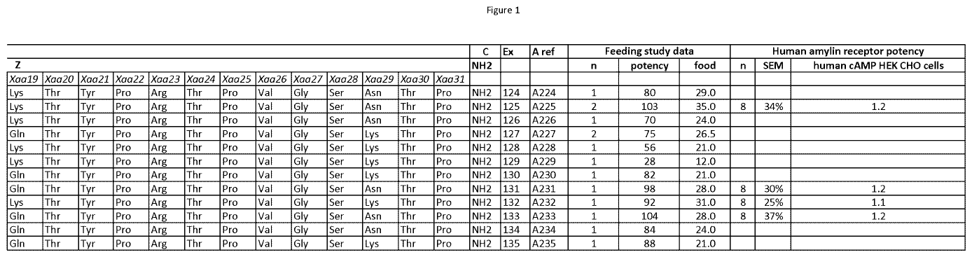

Results of feeding and receptor activity experiments are shown in the right hand columns of the table of FIG. 1.

### SEQUENCES

The amino acid sequences herein are shown with the N-terminus to the left, and where sequences are set out across multiple lines, the N-terminus is to the top left.

Unless indicated otherwise, the amino acid residues in the sequences are L-amino acids.

The amino acid sequences listed in the application are shown using standard letter abbreviations for amino acids.

The specific sequences given herein relate to specific embodiments of the invention.

### DETAILED DESCRIPTION

### Definitions

In order to facilitate review of the various embodiments of this disclosure, the following explanations of specific terms are provided:

Amylin: a 37-peptide hormone. In certain aspects of this disclosure, amylin is may be mammalian amylin, for example rodent or human amylin.

Animal: Living multi-cellular vertebrate organisms, a category that includes, for example, mammals and birds. The term mammal includes both human and non-human mammals. Similarly, the term “subject” includes both human and veterinary subjects. In preferred embodiments of the invention, the subject is a human subject.

Appetite: A natural desire, or longing for food. In one embodiment, appetite is measured by a survey to assess the desire for food. Increased appetite generally leads to increased feeding behaviour.

Appetite Suppressants: Compounds that decrease the desire for food. Commercially available appetite suppressants include, but are not limited to, amfepramone (diethylpropion), phentermine, mazindol and phenylpropanolamine fenfluramine, dexfenfluramine, and fluoxetine.

Body Mass Index (BMI): A mathematical formula for measuring body mass, also sometimes called Quetelet's Index. BMI is calculated by dividing weight (in kg) by height² (in metres²). The current standards for both men and women accepted as “normal” are a BMI of 20-24.9 kg/m². In one embodiment, a BMI of greater than 25 kg/m² can be used to identify an obese subject. Grade I obesity (which is sometimes referred to as being “overweight” rather than obesity) corresponds to a BMI of 25-29.9 kg/m². Grade II obesity corresponds to a BMI of 30-40 kg/m²; and Grade III obesity corresponds to a BMI greater than 40 kg/m² (Jequier, Am. J Clin. Nutr. 45:1035-47, 1987). Ideal body weight will vary among species and individuals based on height, body build, bone structure, and sex.

Diabetes: A failure of cells to transport endogenous glucose across their membranes either because of an endogenous deficiency of insulin and/or a defect in insulin sensitivity. Diabetes is a chronic syndrome of impaired carbohydrate, protein, and fat metabolism owing to insufficient secretion of insulin or to target tissue insulin resistance. It occurs in two major forms: insulin-dependent diabetes mellitus (IDDM, type 1) and non-insulin dependent diabetes mellitus (NIDDM, type II) which differ in etiology, pathology, genetics, age of onset, and treatment.

The two major forms of diabetes are both characterized by an inability to deliver insulin in an amount and with the precise timing that is needed for control of glucose homeostasis. Diabetes type I, or insulin dependent diabetes mellitus (IDDM) is caused by the destruction of β cells, which results in insufficient levels of endogenous insulin. Diabetes type II, or non-insulin dependent diabetes, results from a defect in both the body's sensitivity to insulin, and a relative deficiency in insulin production. Preferred embodiments the invention may relate to treatment of diabetes, including treatment of diabetes type I, or alternatively, diabetes type II. In other embodiments the invention may relate to providing weight loss (including treating or preventing obesity or an overweight condition) in a subject who has diabetes (type I or type II) or a pre-diabetic or other condition or disorder characterised by poor glycaemic control.

Energy Metabolism: The body has to expend a certain amount of energy to maintain normal metabolism. In civilized man this is often taken to be about 2,800 Calories daily. If food consumption does not provide this, weight loss results. However, energy metabolism is also regulated, and, for example, administration of glucagon is thought to increase the metabolic rate so that a greater food intake is required to achieve energy balance and maintain weight. Thus, if food intake is maintained at the usual level, but energy metabolism is increased, weight loss will result.

Food intake: The amount of food consumed by an individual. Food intake can be measured by volume or by weight. For example, food intake may be the total amount of food consumed by an individual. In an animal feeding experiment, ‘Food Intake’ is the weight of a standardised chow consumed by an animal in a 24 hour period. Similar experiments may be conducted on human subjects by providing them with an ab libitum buffet consisting of calorie-controlled portion or items of food and recording how much is consumed (for example by asking them to keep a tally, by using third party observation or by placing each portion or item of food on a separate item of crockery and counting the number of empty items of crockery at the end of the experiment). Or, food intake may be the amount of proteins, fat, carbohydrates, cholesterol, vitamins, minerals, or any other food component, of the individual. “Protein intake” refers to the amount of protein consumed by an individual. Similarly, “fat intake,” “carbohydrate intake,” “cholesterol intake,” “vitamin intake,” and “mineral intake” refer to the amount of proteins, fat, carbohydrates, cholesterol, vitamins, or minerals consumed by an individual.

Normal Daily Diet: The average food intake for an individual of a given species. A normal daily diet can be expressed in terms of caloric intake, protein intake, carbohydrate intake, and/or fat intake. A normal daily diet in humans generally comprises the following: about 2,000, about 2,400, or about 2,800 to significantly more calories. In addition, a normal daily diet in humans generally includes about 12 g to about 45 g of protein, about 120 g to about 610 g of carbohydrate, and about 11 g to about 90 g of fat. A low calorie diet would be no more than about 85%, and preferably no more than about 70%, of the normal caloric intake of a human individual.

In animals, the caloric and nutrient requirements vary depending on the species and size of the animal. For example, in cats, the total caloric intake per pound, as well as the percent distribution of protein, carbohydrate and fat varies with the age of the cat and the reproductive state. A general guideline for cats, however, is 40 cal/lb/day (18.2 cal/kg/day). About 30% to about 40% should be protein, about 7% to about 10% should be from carbohydrate, and about 50% to about 62.5% should be derived from fat intake. One of skill in the art can readily identify the normal daily diet of an individual of any species.

Obesity: A condition in which excess body fat may put a person at health risk (see Barlow and Dietz, Pediatrics 102:E29, 1998; National Institutes of Health, National Heart, Lung, and Blood Institute (NHLBI), Obes. Res. 6 (suppl. 2):51S-209S, 1998). Excess body fat is a result of an imbalance of energy intake and energy expenditure. For example, the Body Mass Index (BMI) may be used to assess obesity. In one commonly used convention, a BMI of 25.0 kg/m² to 29.9 kg/m² is overweight, while a BMI of 30 kg/m² or greater is obese.

In another convention, waist circumference is used to assess obesity. In this convention, in men a waist circumference of 102 cm or more is considered obese, while in women a waist circumference of 89 cm or more is considered obese. Strong evidence shows that obesity affects both the morbidity and mortality of individuals. For example, an obese individual is at increased risk for heart disease, non-insulin dependent (type 2) diabetes, hypertension, stroke, cancer (e.g. endometrial, breast, prostate, and colon cancer), dyslipidemia, gall bladder disease, sleep apnoea, reduced fertility, and osteoarthritis, amongst others (see Lyznicki et al., Am. Fam. Phys. 63:2185, 2001).

Overweight: An individual who weighs more than their ideal body weight. An overweight individual can be obese but is not necessarily obese. For example, an overweight individual is any individual who desires to decrease their weight. In one convention, an overweight individual is an individual with a BMI of 25.0 kg/m² to 29.9 kg/m².

Peripheral administration: Administration outside of the central nervous system. Peripheral administration does not include direct administration to the brain. Peripheral administration includes, but is not limited to intravascular, intramuscular, subcutaneous, inhalation, oral, rectal, transdermal or intra-nasal administration.

Polypeptide: A polymer in which the monomers are amino acid residues which are joined together through amide bonds. A polypeptide may also be referred to as a “peptide”. Unless dictated otherwise by context, the terms “polypeptide”, “peptide”, or “protein” as used herein encompass any amino acid sequence and include modified sequences such as glycoproteins. The terms “polypeptide” and “peptide” covers naturally occurring proteins, as well as those which are recombinantly or synthetically produced. The term “polypeptide fragment” refers to a portion of a polypeptide, for example a fragment which exhibits at least one useful sequence in binding a receptor. The term “functional fragments of a polypeptide” refers to all fragments of a polypeptide that retain an activity of the polypeptide. Biologically functional peptides can also include fusion proteins, in which the peptide of interest has been fused to another peptide that does not decrease its desired activity. In general proteins are longer than peptides/polypeptides. The term “protein” generally being reserved for larger molecules which have either a single chain of 100 or more amino acids or several chains of amino acids joined together.

Subcutaneous administration: Subcutaneous administration is administration of a substance to the subcutaneous layer of fat which is found between the dermis of the skin and the underlying tissue. Subcutaneous administration may be by an injection using a hypodermic needle fitted, for example, to a syringe or a “pen” type injection device. Other administration methods may be used for example microneedles. Injection with a hypodermic needle typically involves a degree of pain on behalf of the recipient. Such pain may be masked by use of a local anaesthetic or analgesic. However, the usual method used to reduce the perceived pain of injections is to merely distract the subject immediately prior to and during the injection. Pain may be minimised by using a relatively small gauge hypodermic needle, by injecting a relatively small volume of substance and by avoiding excessively acidic or alkali compositions which may cause the subject to experience a “stinging” sensation at the injection site. Compositions having a pH of between pH 4 and pH 10 are usually regarded as tolerably comfortable.

Therapeutically effective amount: A dose sufficient to prevent advancement, or to cause regression of a disorder, or which is capable of relieving a sign or symptom of a disorder, or which is capable of achieving a desired result. In some embodiments, a therapeutically effective amount of a compound of the invention is an amount sufficient to inhibit or halt weight gain, or an amount sufficient to decrease appetite.

### Compounds of the Invention

The present inventors have found that example compounds of the invention have properties including causing weight loss in vivo. The compounds also have a long half-life in the blood, meaning that they can be administered at a conveniently low frequency.

Compared with naturally occurring human amylin and previous derivatives of amylin including Pramlintide and Cagrilintide, the compounds of the invention have additional amino acid substituent at one or more positions in the peptide sequence. Additionally, compounds of the invention are lipidated with a fatty dicarboxylic acid. In many embodiments of the invention the fatty dicarboxylic acid is eicosanedioic acid. The fatty dicarboxylic acid (for example the eicosanedioic acid) may be added to the N-terminal of the peptide sequence of the compound via a condensation reaction between one of the acid groups of the fatty dicarboxylic acid and the alpha-amino group at the N-terminal of the peptide sequence of the compound.

Alternatively, the fatty dicarboxylic acid (for example the eicosanedioic acid) may be added to the an additional glutamic acid moeity via a condensation reaction between one of the acid groups of the fatty dicarboxylic acid and the amino group of the glutamic acid, and the glutamic acid may be conjugated in turn to the amino group at the N-terminal of the peptide sequence of the compound via its side chain carboxylic acid group (ie the group attached to the gamma carbon of glutamic acid)

Compounds according to the present invention include sequence substitutions and functionalisations, alone and in novel combinations that have not previously been investigated the beneficial properties found by the current inventors have not previously been seen. According to some embodiments, these chances result in a compound which is up to thrice as potent as Cagrilintide and with a half-life of up to twice as long. Additionally compounds of the invention comprise a peptide portion which is slightly shorter than that present in Cagrilintide. The use of a shorter peptide results in lower cost production and easier purification.

### Peptide Sequences

As described above, compounds of the invention have the formula:

A-B-C.

A is Q, Q-Ser, Q-Glu or Q-Lys, wherein Q is a group of the formula:

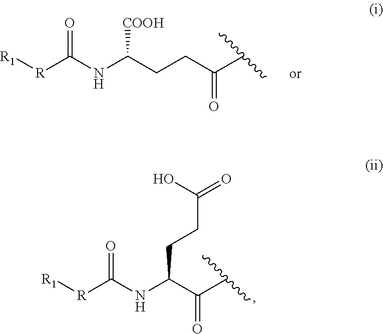

wherein R is a C₈-C₂₈ alkylene or alkenylene chain and R₁ is —CO₂H;
B is a peptide moiety of 32 or 33 amino acid residues, having a sequence of X-Y-Z. wherein:
X is Lys-Cys
Y is Xaa1-Xaa2-Xaa3-Xaa4-Xaa5-Xaa6, wherein;
Xaa1 is Ser or absent
Xaa2 is Asn, Lys, Ser or Thr
Xaa3 is Thr or Leu
Xaa4 is Ala or Ser
Xaa5 is Thr
Xaa6 is Cys
Z is Xaa7-Xaa8-Xaa9-Xaa10-Xaa11-Xaa12-Xaa13-Xaa14-Xaa15-Xaa16-Xaa17-Xaa18-Xaa19-Xaa20-Xaa21-Xaa22-Xaa23-Xaa24-Xaa25-Xaa26-Xaa27-Xaa28-Xaa29-Xaa30-Xaa31 (SEQ ID. NO.: 6), wherein:
Xaa7 is Ala, Val or Met
Xaa8 is Thr or Leu
Xaa9 is Gln or Gly
Xaa10 is Arg or Lys
Xaa11 is Leu
Xaa12 is Ala or Ser
Xaa13 is any amino acid
Xaa14 is Glu, Asp or Phe
Xaa15 is Leu
Xaa16 is His
Xaa17 is Lys or Arg
Xaa18 is Leu
Xaa19 is Gln or Lys
Xaa20 is Thr
Xaa21 is Tyr or Phe
Xaa22 is Pro
Xaa23 is Arg or Lys
Xaa24 is Thr
Xaa25 is Gln, Asp, Pro, Lys or Asn
Xaa26 is Val or Thr
Xaa27 is Gly
Xaa28 is Ser or Ala
Xaa29 is Lys, Asn, Gly, or Asp
Xaa30 is Thr or Ala
Xaa31 is any amino acid
C is a terminal —NH₂ group bound to the C terminal of peptide moiety B.

As can be seen, a substantial portion of the compound consists of peptide sequence B. This may be 32 or 33 amino acid residues in length. It is preferably 32 amino acids in length.

Considering each of the residues of B in turn,
B has a sequence of X-Y-Z, and X is Lys-Cys
Y is Xaa1-Xaa2-Xaa3-Xaa4-Xaa5-Xaa6.
Xaa1 is Ser or absent. In preferred embodiments Xaa1 is absent. That results in peptide sequence B being 32 amino acid residues in length.
Xaa2 is Asn, Lys, Ser or Thr. Preferably Xaa2 is Asn, Lys or Ser.
Xaa3 is Thr or Leu. Preferably Xaa3 is Thr when Xaa1 is absent. Preferably, Xaa3 is Leu when Xaa1 is Ser.
Xaa4 is Ala or Ser. Preferably Xaa4 is Ala when Xaa1 is absent. Preferably Xaa2 is Ser when Xaa1 is Ser.
Xaa5 is Thr
Xaa6 is Cys

Preferably Xaa1 is absent, and Xaa2 is Asn, Lys or Ser, and Xaa3 is Thr, and Xaa4 is Ala, and Xaa5 is Thr and Xaa6 is Cys.

Z is Xaa7-Xaa8-Xaa9-Xaa10-Xaa11-Xaa12-Xaa13-Xaa14-Xaa15-Xaa16-Xaa17-Xaa18-Xaa19-Xaa20-Xaa21-Xaa22-Xaa23-Xaa24-Xaa25-Xaa26-Xaa27-Xaa28-Xaa29-Xaa30-Xaa31 (SEQ ID. NO.: 6), wherein:
Xaa7 is Ala, Val or Met. Preferably Xaa7 is Ala.
Xaa8 is Thr or Leu. Preferably Xaa8 is Thr.
Xaa9 is Gln, Gly. Preferably Xaa9 is Gln.
Xaa10 is Arg or Lys. Preferably Xaa10 is Arg.
Xaa11 is Leu.
Xaa12 is Ala or Ser. Preferably Xaa12 is Ala.

Preferably Xaa7 to Xaa12 is Ala-Thr-Gln-Arg-Leu-Ala (SEQ ID. NO.: 7),

Preferably Xaa1 is absent, and Xaa2 is Asn, Lys or Ser, and Xaa3 is Thr, and Xaa4 is Ala, and Xaa5 is Thr and Xaa6 is Cys, and Xaa7 to Xaa12 is Ala-Thr-Gln-Arg-Leu-Ala (SEQ ID. NO.: 7).
Xaa13 is any amino acid. Preferably Xaa13 is Glu, Asp, Gln, Asn or Lys, more preferably Xaa13 is Asp, Glu or Gln.
Xaa14 is Glu, Asp or Phe. Preferably Xaa14 is Glu.
Xaa15 is Leu.
Xaa16 is His.
Xaa17 is Lys or Arg. Preferably Xaa17 is Lys.
Xaa18 is Leu.
Xaa19 is Gln or Lys. Preferably Xaa19 is Gln.
Xaa20 is Thr.
Xaa21 is Tyr or Phe. Preferably Xaa21 is Tyr
Xaa22 is Pro.
Xaa23 is Arg or Lys. Preferably Xaa23 is Arg
Xaa24 is Thr or Lys. Preferably Xaa24 is Thr.

Preferably, Xaa14 to Xaa 24 is Glu-Leu-His-Lys-Leu-Gln-Thr-Tyr-Pro-Arg-Thr (SEQ ID. NO.: 8). Alternatively, Xaa14 to Xaa 24 is Glu-Leu-His-Lys-Leu-Lys-Thr-Tyr-Pro-Arg-Thr (SEQ ID. NO.: 9).

Preferably, Xaa13 is Glu, Asp, Gln, Asn or Lys (more preferably Xaa13 is Asp, Glu or Gln), and Xaa14 to Xaa 24 is Glu-Leu-His-Lys-Leu-Gln-Thr-Tyr-Pro-Arg-Thr (SEQ ID. NO.: 8).

Alternatively, Xaa13 is Glu, Asp, Gln, Asn or Lys (more preferably Xaa13 is Asp, Glu or Gln), and Xaa14 to Xaa 24 is Glu-Leu-His-Lys-Leu-Lys-Thr-Tyr-Pro-Arg-Thr (SEQ ID. NO.: 9).

Preferably, Xaa1 is absent, and Xaa2 is Asn, Lys or Ser, and Xaa3 is Thr, and Xaa4 is Ala, and Xaa5 is Thr and Xaa6 is Cys, and Xaa7 to Xaa12 is Ala-Thr-Gln-Arg-Leu-Ala (SEQ ID. NO.: 7) and Xaa14 to Xaa 24 is Glu-Leu-His-Lys-Leu-Gln-Thr-Tyr-Pro-Arg-Thr (SEQ ID. NO.: 8).

Alternatively, Xaa1 is absent, and Xaa2 is Asn, Lys or Ser, and Xaa3 is Thr, and Xaa4 is Ala, and Xaa5 is Thr and Xaa6 is Cys, and Xaa7 to Xaa12 is Ala-Thr-Gln-Arg-Leu-Ala (SEQ ID. NO.: 7) and Xaa14 to Xaa 24 is Glu-Leu-His-Lys-Leu-Lys-Thr-Tyr-Pro-Arg-Thr (SEQ ID. NO.: 9).

Preferably, Xaa1 is absent, and Xaa2 is Asn, Lys or Ser, and Xaa3 is Thr, and Xaa4 is Ala, and Xaa5 is Thr and Xaa6 is Cys, and Xaa7 to Xaa12 is Ala-Thr-Gln-Arg-Leu-Ala (SEQ ID. NO.: 7) and Xaa13 is Glu, Asp, Gln, Asn or Lys (more preferably Xaa13 is Asp, Glu or Gln), and Xaa14 to Xaa 24 is Glu-Leu-His-Lys-Leu-Gln-Thr-Tyr-Pro-Arg-Thr (SEQ ID. NO.: 8).

Alternatively, Xaa1 is absent, and Xaa2 is Asn, Lys or Ser, and Xaa3 is Thr, and Xaa4 is Ala, and Xaa5 is Thr and Xaa6 is Cys, and Xaa7 to Xaa12 is Ala-Thr-Gln-Arg-Leu-Ala (SEQ ID. NO.: 7) and Xaa13 is Glu, Asp, Gln, Asn or Lys (more preferably Xaa13 is Asp, Glu or Gln), and Xaa14 to Xaa 24 is Glu-Leu-His-Lys-Leu-Lys-Thr-Tyr-Pro-Arg-Thr (SEQ ID. NO.: 8).

Preferably, Xaa1 is absent, and Xaa2 is Asn, Lys or Ser, and Xaa3 is Thr, and Xaa4 is Ala, and Xaa5 is Thr and Xaa6 is Cys, and and Xaa14 to Xaa 24 is Glu-Leu-His-Lys-Leu-Gln-Thr-Tyr-Pro-Arg-Thr (SEQ ID. NO.: 8).

Alternatively, Xaa1 is absent, and Xaa2 is Asn, Lys or Ser, and Xaa3 is Thr, and Xaa4 is Ala, and Xaa5 is Thr and Xaa6 is Cys, and Xaa14 to Xaa 24 is Glu-Leu-His-Lys-Leu-Lys-Thr-Tyr-Pro-Arg-Thr (SEQ ID. NO.: 9).

Preferably, Xaa1 is absent, and Xaa2 is Asn, Lys or Ser, and Xaa3 is Thr, and Xaa4 is Ala, and Xaa5 is Thr and Xaa6 is Cys, and Xaa13 is Glu, Asp, Gln, Asn or Lys (more preferably Xaa13 is Asp, Glu or Gln), and Xaa14 to Xaa 24 is Glu-Leu-His-Lys-Leu-Gln-Thr-Tyr-Pro-Arg-Thr (SEQ ID. NO.: 8).

Alternatively, Xaa1 is absent, and Xaa2 is Asn, Lys or Ser, and Xaa3 is Thr, and Xaa4 is Ala, and Xaa5 is Thr and Xaa6 is Cys, and Xaa13 is Glu, Asp, Gln, Asn or Lys (more preferably Xaa13 is Asp, Glu or Gln), and Xaa14 to Xaa 24 is Glu-Leu-His-Lys-Leu-Lys-Thr-Tyr-Pro-Arg-Thr (SEQ ID. NO.: 9).

Preferably, Xaa7 to Xaa12 is Ala-Thr-Gln-Arg-Leu-Ala (SEQ ID. NO.: 7) and Xaa14 to Xaa 24 is Glu-Leu-His-Lys-Leu-Gln-Thr-Tyr-Pro-Arg-Thr (SEQ ID. NO.: 8).

Alternatively, Xaa7 to Xaa12 is Ala-Thr-Gln-Arg-Leu-Ala (SEQ ID. NO.: 7) and Xaa14 to Xaa 24 is Glu-Leu-His-Lys-Leu-Lys-Thr-Tyr-Pro-Arg-Thr (SEQ ID. NO.: 9).

Preferably, Xaa7 to Xaa12 is Ala-Thr-Gln-Arg-Leu-Ala (SEQ ID. NO.: 7) and Xaa13 is Glu, Asp, Gln, Asn or Lys (more preferably Xaa13 is Asp, Glu or Gln), and Xaa14 to Xaa 24 is Glu-Leu-His-Lys-Leu-Gln-Thr-Tyr-Pro-Arg-Thr (SEQ ID. NO.: 8).

Alternatively, Xaa7 to Xaa12 is Ala-Thr-Gln-Arg-Leu-Ala (SEQ ID. NO.: 7) and Xaa13 is Glu, Asp, Gln, Asn or Lys (more preferably Xaa13 is Asp, Glu or Gln), and Xaa14 to Xaa 24 is Glu-Leu-His-Lys-Leu-Lys-Thr-Tyr-Pro-Arg-Thr (SEQ ID. NO.: 9).
Xaa25 is Pro, Lys or Asn. Preferably Xaa25 is Pro. In alternative embodiments it may be Lys.
Xaa26 is Val or Thr. Preferably, Xaa26 is Val. In alternative embodiments it may be Thr.
Xaa27 is Gly or Val. Preferably Xaa27 is Gly.
Xaa28 is Ser or Gly. Preferably Xaa28 is Ser.

Preferably, Xaa27-Xaa28 is Gly-Ser.

Preferably, Xaa14 to Xaa 24 is Glu-Leu-His-Lys-Leu-Gln-Thr-Tyr-Pro-Arg-Thr (SEQ ID. NO.: 8). Alternatively, Xaa14 to Xaa 24 is Glu-Leu-His-Lys-Leu-Lys-Thr-Tyr-Pro-Arg-Thr (SEQ ID. NO.: 9) and Xaa27-Xaa28 is Gly-Ser.

Preferably, Xaa13 is Glu, Asp, Gln, Asn or Lys (more preferably Xaa13 is Asp, Glu or Gln), and Xaa14 to Xaa 24 is Glu-Leu-His-Lys-Leu-Gln-Thr-Tyr-Pro-Arg-Thr (SEQ ID. NO.: 8) and Xaa27-Xaa28 is Gly-Ser.

Alternatively, Xaa13 is Glu, Asp, Gln, Asn or Lys (more preferably Xaa13 is Asp, Glu or Gln), and Xaa14 to Xaa 24 is Glu-Leu-His-Lys-Leu-Lys-Thr-Tyr-Pro-Arg-Thr (SEQ ID. NO.: 9) and Xaa27-Xaa28 is Gly-Ser.

Preferably, Xaa1 is absent, and Xaa2 is Asn, Lys or Ser, and Xaa3 is Thr, and Xaa4 is Ala, and Xaa5 is Thr and Xaa6 is Cys, and Xaa7 to Xaa12 is Ala-Thr-Gln-Arg-Leu-Ala (SEQ ID. NO.: 7) and Xaa14 to Xaa 24 is Glu-Leu-His-Lys-Leu-Gln-Thr-Tyr-Pro-Arg-Thr (SEQ ID. NO.: 8) and Xaa27-Xaa28 is Gly-Ser.

Alternatively, Xaa1 is absent, and Xaa2 is Asn, Lys or Ser, and Xaa3 is Thr, and Xaa4 is Ala, and Xaa5 is Thr and Xaa6 is Cys, and Xaa7 to Xaa12 is Ala-Thr-Gln-Arg-Leu-Ala (SEQ ID. NO.: 7) and Xaa14 to Xaa 24 is Glu-Leu-His-Lys-Leu-Lys-Thr-Tyr-Pro-Arg-Thr (SEQ ID. NO.: 9) and Xaa27-Xaa28 is Gly-Ser.

Preferably, Xaa1 is absent, and Xaa2 is Asn, Lys or Ser, and Xaa3 is Thr, and Xaa4 is Ala, and Xaa5 is Thr and Xaa6 is Cys, and Xaa7 to Xaa12 is Ala-Thr-Gln-Arg-Leu-Ala (SEQ ID. NO.: 7) and Xaa13 is Glu, Asp, Gln, Asn or Lys (more preferably Xaa13 is Asp, Glu or Gln), and Xaa14 to Xaa 24 is Glu-Leu-His-Lys-Leu-Gln-Thr-Tyr-Pro-Arg-Thr (SEQ ID. NO.: 8) and Xaa27-Xaa28 is Gly-Ser.

Alternatively, Xaa1 is absent, and Xaa2 is Asn, Lys or Ser, and Xaa3 is Thr, and Xaa4 is Ala, and Xaa5 is Thr and Xaa6 is Cys, and Xaa7 to Xaa12 is Ala-Thr-Gln-Arg-Leu-Ala (SEQ ID. NO.: 7) and Xaa13 is Glu, Asp, Gln, Asn or Lys (more preferably Xaa13 is Asp, Glu or Gln), and Xaa14 to Xaa 24 is Glu-Leu-His-Lys-Leu-Lys-Thr-Tyr-Pro-Arg-Thr (SEQ ID. NO.: 9) and Xaa27-Xaa28 is Gly-Ser.

Preferably, Xaa1 is absent, and Xaa2 is Asn, Lys or Ser, and Xaa3 is Thr, and Xaa4 is Ala, and Xaa5 is Thr and Xaa6 is Cys, and and Xaa14 to Xaa 24 is Glu-Leu-His-Lys-Leu-Gln-Thr-Tyr-Pro-Arg-Thr (SEQ ID. NO.: 8) and Xaa27-Xaa28 is Gly-Ser.

Alternatively, Xaa1 is absent, and Xaa2 is Asn, Lys or Ser, and Xaa3 is Thr, and Xaa4 is Ala, and Xaa5 is Thr and Xaa6 is Cys, and Xaa14 to Xaa 24 is Glu-Leu-His-Lys-Leu-Lys-Thr-Tyr-Pro-Arg-Thr (SEQ ID. NO.: 9) and Xaa27-Xaa28 is Gly-Ser.

Preferably, Xaa1 is absent, and Xaa2 is Asn, Lys or Ser, and Xaa3 is Thr, and Xaa4 is Ala, and Xaa5 is Thr and Xaa6 is Cys, and Xaa13 is Glu, Asp, Gln, Asn or Lys (more preferably Xaa13 is Asp, Glu or Gln), and Xaa14 to Xaa 24 is Glu-Leu-His-Lys-Leu-Gln-Thr-Tyr-Pro-Arg-Thr (SEQ ID. NO.: 8) and Xaa27-Xaa28 is Gly-Ser.

Alternatively, Xaa1 is absent, and Xaa2 is Asn, Lys or Ser, and Xaa3 is Thr, and Xaa4 is Ala, and Xaa5 is Thr and Xaa6 is Cys, and Xaa13 is Glu, Asp, Gln, Asn or Lys (more preferably Xaa13 is Asp, Glu or Gln), and Xaa14 to Xaa 24 is Glu-Leu-His-Lys-Leu-Lys-Thr-Tyr-Pro-Arg-Thr (SEQ ID. NO.: 9) and Xaa27-Xaa28 is Gly-Ser.

Preferably, Xaa7 to Xaa12 is Ala-Thr-Gln-Arg-Leu-Ala and Xaa14 to Xaa 24 is Glu-Leu-His-Lys-Leu-Gln-Thr-Tyr-Pro-Arg-Thr (SEQ ID. NO.: 8) and Xaa27-Xaa28 is Gly-Ser.

Alternatively, Xaa7 to Xaa12 is Ala-Thr-Gln-Arg-Leu-Ala (SEQ ID. NO.: 7) and Xaa14 to Xaa 24 is Glu-Leu-His-Lys-Leu-Lys-Thr-Tyr-Pro-Arg-Thr (SEQ ID. NO.: 9) and Xaa27-Xaa28 is Gly-Ser.

Preferably, Xaa7 to Xaa12 is Ala-Thr-Gln-Arg-Leu-Ala (SEQ ID. NO.: 7) and Xaa13 is Glu, Asp, Gln, Asn or Lys (more preferably Xaa13 is Asp, Glu or Gln), and Xaa14 to Xaa 24 is Glu-Leu-His-Lys-Leu-Gln-Thr-Tyr-Pro-Arg-Thr (SEQ ID. NO.: 8) and Xaa27-Xaa28 is Gly-Ser.

Alternatively, Xaa7 to Xaa12 is Ala-Thr-Gln-Arg-Leu-Ala (SEQ ID. NO.: 7) and Xaa13 is Glu, Asp, Gln, Asn or Lys (more preferably Xaa13 is Asp, Glu or Gln), and Xaa14 to Xaa 24 is Glu-Leu-His-Lys-Leu-Lys-Thr-Tyr-Pro-Arg-Thr (SEQ ID. NO.: 9) and Xaa27-Xaa28 is Gly-Ser.
Xaa29 is Lys, Asn, Gly, Ala, Asp. Preferably, Xaa29 is Lys.
Xaa30 is Thr or Asn. Preferably, Xaa30 is Thr.
Xaa31 is any amino acid. Preferably, Xaa31 is Pro, Ala, Leu, Ile, Val, Ser, or Gln (most preferably Pro).
Xaa30-Xaa31 is preferably Thr-Pro.

Preferably, Xaa14 to Xaa 24 is Glu-Leu-His-Lys-Leu-Gln-Thr-Tyr-Pro-Arg-Thr (SEQ ID. NO.: 8). Alternatively, Xaa14 to Xaa 24 is Glu-Leu-His-Lys-Leu-Lys-Thr-Tyr-Pro-Arg-Thr (SEQ ID. NO.: 9) and Xaa27-Xaa28 is Gly-Ser, and Xaa30 is Thr, and Xaa31 is Pro, Ala, Leu, Ile, Val, Ser, or Gln (most preferably Pro).

Preferably, Xaa13 is Glu, Asp, Gln, Asn or Lys (more preferably Xaa13 is Asp, Glu or Gln), and Xaa14 to Xaa 24 is Glu-Leu-His-Lys-Leu-Gln-Thr-Tyr-Pro-Arg-Thr (SEQ ID. NO.: 8) and Xaa27-Xaa28 is Gly-Ser, and Xaa30 is Thr, and Xaa31 is Pro, Ala, Leu, Ile, Val, Ser, or Gln (most preferably Pro).

Alternatively, Xaa13 is Glu, Asp, Gln, Asn or Lys (more preferably Xaa13 is Asp, Glu or Gln), and Xaa14 to Xaa 24 is Glu-Leu-His-Lys-Leu-Lys-Thr-Tyr-Pro-Arg-Thr (SEQ ID. NO.: 9) and Xaa27-Xaa28 is Gly-Ser, and Xaa30 is Thr, and Xaa31 is Pro, Ala, Leu, Ile, Val, Ser, or Gln (most preferably Pro).

Preferably, Xaa1 is absent, and Xaa2 is Asn, Lys or Ser, and Xaa3 is Thr, and Xaa4 is Ala, and Xaa5 is Thr and Xaa6 is Cys, and Xaa7 to Xaa12 is Ala-Thr-Gln-Arg-Leu-Ala (SEQ ID. NO.: 7) and Xaa14 to Xaa 24 is Glu-Leu-His-Lys-Leu-Gln-Thr-Tyr-Pro-Arg-Thr (SEQ ID. NO.: 8) and Xaa27-Xaa28 is Gly-Ser, and Xaa30 is Thr, and Xaa31 is Pro, Ala, Leu, Ile, Val, Ser, or Gln (most preferably Pro).

Alternatively, Xaa1 is absent, and Xaa2 is Asn, Lys or Ser, and Xaa3 is Thr, and Xaa4 is Ala, and Xaa5 is Thr and Xaa6 is Cys, and Xaa7 to Xaa12 is Ala-Thr-Gln-Arg-Leu-Ala (SEQ ID. NO.: 7) and Xaa14 to Xaa 24 is Glu-Leu-His-Lys-Leu-Lys-Thr-Tyr-Pro-Arg-Thr (SEQ ID. NO.: 9) and Xaa27-Xaa28 is Gly-Ser, and Xaa30 is Thr, and Xaa31 is Pro, Ala, Leu, Ile, Val, Ser, or Gln (most preferably Pro).

Preferably, Xaa1 is absent, and Xaa2 is Asn, Lys or Ser, and Xaa3 is Thr, and Xaa4 is Ala, and Xaa5 is Thr and Xaa6 is Cys, and Xaa7 to Xaa12 is Ala-Thr-Gln-Arg-Leu-Ala (SEQ ID. NO.: 7) and Xaa13 is Glu, Asp, Gln, Asn or Lys (more preferably Xaa13 is Asp, Glu or Gln), and Xaa14 to Xaa 24 is Glu-Leu-His-Lys-Leu-Gln-Thr-Tyr-Pro-Arg-Thr (SEQ ID. NO.: 8) and Xaa27-Xaa28 is Gly-Ser, and Xaa30 is Thr, and Xaa31 is Pro, Ala, Leu, Ile, Val, Ser, or Gln (most preferably Pro).

Alternatively, Xaa1 is absent, and Xaa2 is Asn, Lys or Ser, and Xaa3 is Thr, and Xaa4 is Ala, and Xaa5 is Thr and Xaa6 is Cys, and Xaa7 to Xaa12 is Ala-Thr-Gln-Arg-Leu-Ala (SEQ ID. NO.: 7) and Xaa13 is Glu, Asp, Gln, Asn or Lys (more preferably Xaa13 is Asp, Glu or Gln), and Xaa14 to Xaa 24 is Glu-Leu-His-Lys-Leu-Lys-Thr-Tyr-Pro-Arg-Thr (SEQ ID. NO.: 9) and Xaa27-Xaa28 is Gly-Ser, and Xaa30 is Thr, and Xaa31 is Pro, Ala, Leu, Ile, Val, Ser, or Gln (most preferably Pro).

Preferably, Xaa1 is absent, and Xaa2 is Asn, Lys or Ser, and Xaa3 is Thr, and Xaa4 is Ala, and Xaa5 is Thr and Xaa6 is Cys, and Xaa14 to Xaa 24 is Glu-Leu-His-Lys-Leu-Gln-Thr-Tyr-Pro-Arg-Thr (SEQ ID. NO.: 8) and Xaa27-Xaa28 is Gly-Ser, and Xaa30 is Thr, and Xaa31 is Pro, Ala, Leu, Ile, Val, Ser, or Gln (most preferably Pro).

Alternatively, Xaa1 is absent, and Xaa2 is Asn, Lys or Ser, and Xaa3 is Thr, and Xaa4 is Ala, and Xaa5 is Thr and Xaa6 is Cys, and Xaa14 to Xaa 24 is Glu-Leu-His-Lys-Leu-Lys-Thr-Tyr-Pro-Arg-Thr (SEQ ID. NO.: 9) and Xaa27-Xaa28 is Gly-Ser, and Xaa30 is Thr, and Xaa31 is Pro, Ala, Leu, Ile, Val, Ser, or Gln (most preferably Pro).

Preferably, Xaa1 is absent, and Xaa2 is Asn, Lys or Ser, and Xaa3 is Thr, and Xaa4 is Ala, and Xaa5 is Thr and Xaa6 is Cys, and Xaa13 is Glu, Asp, Gln, Asn or Lys (more preferably Xaa13 is Asp, Glu or Gln), and Xaa14 to Xaa 24 is Glu-Leu-His-Lys-Leu-Gln-Thr-Tyr-Pro-Arg-Thr (SEQ ID. NO.: 8) and Xaa27-Xaa28 is Gly-Ser, and Xaa30 is Thr, and Xaa31 is Pro, Ala, Leu, Ile, Val, Ser, or Gln (most preferably Pro).

Alternatively, Xaa1 is absent, and Xaa2 is Asn, Lys or Ser, and Xaa3 is Thr, and Xaa4 is Ala, and Xaa5 is Thr and Xaa6 is Cys, and Xaa13 is Glu, Asp, Gln, Asn or Lys (more preferably Xaa13 is Asp, Glu or Gln), and Xaa14 to Xaa 24 is Glu-Leu-His-Lys-Leu-Lys-Thr-Tyr-Pro-Arg-Thr (SEQ ID. NO.: 9) and Xaa27-Xaa28 is Gly-Ser, and Xaa30 is Thr, and Xaa31 is Pro, Ala, Leu, Ile, Val, Ser, or Gln (most preferably Pro).

Preferably, Xaa7 to Xaa12 is Ala-Thr-Gln-Arg-Leu-Ala (SEQ ID. NO.: 7) and Xaa14 to Xaa 24 is Glu-Leu-His-Lys-Leu-Gln-Thr-Tyr-Pro-Arg-Thr (SEQ ID. NO.: 8) and Xaa27-Xaa28 is Gly-Ser, and Xaa30 is Thr, and Xaa31 is Pro, Ala, Leu, Ile, Val, Ser, or Gln (most preferably Pro).

Alternatively, Xaa7 to Xaa12 is Ala-Thr-Gln-Arg-Leu-Ala (SEQ ID. NO.: 7) and Xaa14 to Xaa 24 is Glu-Leu-His-Lys-Leu-Lys-Thr-Tyr-Pro-Arg-Thr (SEQ ID. NO.: 9) and Xaa27-Xaa28 is Gly-Ser, and Xaa30 is Thr, and Xaa31 is Pro, Ala, Leu, Ile, Val, Ser, or Gln (most preferably Pro).

Preferably, Xaa7 to Xaa12 is Ala-Thr-Gln-Arg-Leu-Ala (SEQ ID. NO.: 7) and Xaa13 is Glu, Asp, Gln, Asn or Lys (more preferably Xaa13 is Asp, Glu or Gln), and Xaa14 to Xaa 24 is Glu-Leu-His-Lys-Leu-Gln-Thr-Tyr-Pro-Arg-Thr (SEQ ID. NO.: 8) and Xaa27-Xaa28 is Gly-Ser, and Xaa30 is Thr, and Xaa31 is Pro, Ala, Leu, Ile, Val, Ser, or Gln (most preferably Pro).

Alternatively, Xaa7 to Xaa12 is Ala-Thr-Gln-Arg-Leu-Ala (SEQ ID. NO.: 7) and Xaa13 is Glu, Asp, Gln, Asn or Lys (more preferably Xaa13 is Asp, Glu or Gln), and Xaa14 to Xaa 24 is Glu-Leu-His-Lys-Leu-Lys-Thr-Tyr-Pro-Arg-Thr (SEQ ID. NO.: 9) and Xaa27-Xaa28 is Gly-Ser, and Xaa30 is Thr, and Xaa31 is Pro, Ala, Leu, Ile, Val, Ser, or Gln (most preferably Pro).

### Group A and Q

A is Q, Q-Ser, Q-Glu or Q-Lys. Preferably A is Q, Q-Ser or Q-Lys
Q is a moiety of the formula:

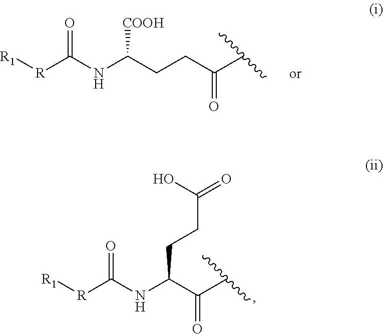

wherein R is a C₈-C₂₈ alkylene or alkenylene chain and R₁ is —CO₂H.

Within the Q portion of the compound, group R is an alkylene or alkenylene chain, which is linked at one end to the B portion of the molecule (or when A is Q-Ser to the Ser residue of A, or when A is Q-Lys to the Lys residue of A) by the Glu residue part of the Q portion of the molecule via an amide bond. At its other end, the R alkylene or alkenylene chain is linked to the R₁ acid group (CO₂H).

Option (i) above represents the situation wherein the Glu residue of Q is attached to the B portion of the compound (or when A is Q-Ser to the Ser residue of A, or when A is Q-Lys to the Lys residue of A) through the γ-carboxylic acid group of Glu.

Option (ii) above represents the situation wherein the Glu residue in the Q portion of the compound is attached to the B portion of the compound (or when A is Q-Ser to the Ser residue of A, or when A is Q-Lys to the Lys residue of A) through the α-carboxylic acid group of Glu. In a preferred embodiment of the invention, the Q portion of the compound is of formula (i), i.e. the Glu residue in the Q portion is attached to the rest of the compound through the γ-carboxylic acid group of the Glu residue portion of Q.

Generally, R has an even number of carbon atoms. For example, R can be an alkylene or alkenylene chain that is found in naturally-occurring fatty acids. The root fatty acid has a chain length two higher than the number of carbon atoms in the R an alkylene or alkenylene chain.

R is preferably a C₁₆-C₁₈ alkylene or alkenylene group. For example, R is preferably a straight chain alkylene or alkenylene group. For example, R may be a C₁₆ or C₁₈ straight chain alkylene group. For example, when R is a C₁₆, group, it can be provided by an octadecanedioic acid moiety. For example, when R is a C₁₈, group, it can be provided by an eicosanedioic acid moiety.

In a preferred embodiment, R is a C₁₈ alkylene group.

In the table of FIG. 1 specific identities of the dicarboxylic acid portion of Q are given by reference to the root fatty dicarboxylic acid constituting part of Q. That is to say that when “eicosanedioic acid” appears in the table of FIG. 1, R is a C₁₈ straight chain alkylene group. The glutamic acid portion of Q is identified by the “γGlu” or “Glu” symbol. “γGlu” signifying that the Q portion of the compound accords to formula (i). “Glu” signifying that the Q portion of the compound accords to formula (ii).

In especially preferred embodiments, the compound of the invention is one of the specific compounds of the invention set out in the table of FIG. 1.

### Disulphide Bridge

In all embodiments of the invention the compound of formula A-B-C has a Cys residue close to the N terminal of the peptide sequence as indicated and a further Cys residue 5 (or occasionally 6) residues along the peptide portion of the molecule towards the C terminal. Cys residues comprise a side chain thiol group, which may be oxidised to form a disulphide —S—S— linkage between the two Cys residues. Whilst the non-disulphide-linked versions of compounds of the invention are of value (for example as intermediate compounds in the production of disulphide-linked versions of compounds of the invention, the presence of this —S—S— linkage is essential for biological activity and is therefore a preferred feature according to certain embodiments of the invention. The compounds set out in the table of FIG. 1 have all been made with a —S—S— linkage between the two Cys residues.

### Derivatives and Salts

The present invention provides compounds of formula A-B-C, derivatives of such compounds, and salts or solvates of such compounds and derivatives.

The compounds, derivatives and salts may be produced by recombinant methods which are well-known in the art or alternatively they may be produced by synthetic methods, again which are well-known in the art.

### Derivatives

Whilst in some embodiments, the invention relates directly to a compound of formula A-B-C and is not a derivative, in other embodiments the invention relates to a derivative of a compound of formula A-B-C. The derivative may for example comprise one or more derivatisations selected from amidation, glycosylation, carbamylation, acylation, sulfation, phosphorylation, additional cyclization, additional lipidization, pegylation and fusion to another peptide or protein to form a fusion protein, for example the derivative may comprise one or more derivatisations selected from amidation, glycosylation, carbamylation, acylation, sulfation, phosphorylation, cyclization, lipidization and pegylation. The structure may be modified at random positions within the molecule, or at predetermined positions within the molecule and may include one, two, three or more attached chemical moieties. According to certain embodiments the term “derivative” does not extend to compounds wherein the peptide sequence is changed.

The derivative may for example be a fusion protein, whereby the A-B-C structure of a compound of the invention is fused to another protein or polypeptide (the fusion partner) using recombinant methods known in the art. Alternatively, such a fusion protein may be synthetically synthesized by any known method. Such a fusion protein comprises the structure of formula A-B-C. Any suitable peptide or protein can be used as the fusion partner (e.g., serum albumin, carbonic anhydrase, glutathione-S-transferase or thioredoxin, etc.). Such fusion proteins may be made by linking the carboxy-terminus of the fusion partner to the amino-terminus of the structure of formula A-B-C or vice versa. Optionally, a cleavable linker may be used to link the structure of formula A-B-C to the fusion partner. A resulting cleavable fusion protein may be cleaved in vivo such that an active form of a compound of the invention is released. Examples of such cleavable linkers include, but are not limited to, the linkers Asp-Asp-Asp-Asp-Tyr (SEQ ID NO: 10), Gly-Pro-Arg, Ala-Gly-Gly and His-Pro-Phe-His-Leu (SEQ ID NO: 11), which can be cleaved by enterokinase, thrombin, ubiquitin cleaving enzyme and renin, respectively. For details, see for example U.S. Pat. No. 6,410,707, the contents of which are incorporated herein by reference.

A derivative of the invention may for example be a physiologically functional derivative of the structure of formula A-B-C. The term “physiologically functional derivative” is used herein to denote a chemical derivative of a compound of formula A-B-C having the same physiological function as the corresponding unmodified compound. For example, a physiologically functionally derivative may be convertible in the body to a compound of formula A-B-C. According to the present invention, examples of physiologically functional derivatives include esters, amides, and carbamates; preferably esters and amides.

For example, pharmaceutically acceptable esters and amides of the compounds of the invention may comprise a C1-20 alkyl-, C2-20 alkenyl-, C5-10 aryl-, C5-10 ar-C1-20 alkyl-, or amino acid-ester group or amide group attached at an appropriate site, for example formed by reaction of an alkyl, alkenyl aryl, aralkyl or amino alkyl group containing an alcohol or amino moiety with an acid moiety present in the compound of formula A-B-C, or formed by reaction of an alkyl, alkenyl aryl, aralkyl or amino alkyl group containing an activated acyl group with an alcohol or amine group present in the compound of formula A-B-C. Examples of suitable moieties are hydrophobic substituents with 4 to 26 carbon atoms, preferably 5 to 19 carbon atoms. Suitable lipid groups include fatty acids (e.g. lauroyl (C₁₂H₂₃), palmityl (C₁₅H₃₁), oleyl (C₁₅H₂₉) or stearyl (C₁₇H₃₅)) and bile acids (e.g. cholate or deoxycholate).

Lipidisation (both in relation to compounds of the invention and derivatives of those compounds) markedly increases the absorption of the compounds relative to the rate of absorption of the corresponding unlipidised compounds, as well as prolonging blood and tissue retention of the compounds. Suitable lipid-containing moieties are hydrophobic substituents with 4 to 26 carbon atoms, preferably 5 to 19 carbon atoms. Suitable lipid groups include fatty acids (e.g. lauroyl (C₁₂H₂₃), palmityl (C₁₅H₃₁), oleyl (C₁₅H₂₉) or stearyl (C₁₇H₃₅)) and bile acids (e.g. cholate or deoxycholate). Whilst lipid functionalised compounds of the invention may have benefits in certain situations, it is expected that in most cases, it will be preferred if a compound of the invention is not further derivatised, such that there are not additional lipid groups present beyond the fatty dicarboxylic acid portion of the compound according to the formula A-B-C.

Cyclization methods (in the context of both compounds of the invention and also derivatives of those compounds) include cyclization through the formation of a disulfide bridge, and head-to-tail cyclization using a cyclization resin. Cyclized peptides may have enhanced stability, including increased resistance to enzymatic degradation, as a result of their conformational constraints. Cyclization may in particular be expedient where the uncyclized peptide includes an N-terminal cysteine group. Suitable cyclized peptides include monomeric and dimeric head-to-tail cyclized structures. Cyclized peptides may include one or more additional residues, especially an additional cysteine incorporated for the purpose of formation of a disulfide bond or a side chain incorporated for the purpose of resin-based cyclization. Whilst additional cyclisation may have benefits in certain situations, it is expected that in most cases, it will be preferred if a compound of the invention is not further cyclied, such that there is not additional cyclisation present beyond that achieved by the —S—S— between the two cysteine residues according to the compound of formula A-B-C.

The derivative may for example be a PEGylated structure of formula A-B-C. Derivatives which are PEGylated compounds of the invention may provide additional advantages such as increased solubility, stability and circulating time of the polypeptide, or decreased immunogenicity (see U.S. Pat. No. 4,179,337, the contents of which are incorporated herein by reference).

Chemical moieties for derivatisation of a compound of the invention may also be selected from water soluble polymers such as polyethylene glycol, ethylene glycol/propylene glycol copolymers, carboxymethylcellulose, dextran, polyvinyl alcohol and the like. A polymer moiety for derivatisation of a compound of the invention may be of any molecular weight and may be branched or unbranched. For ease in handling and manufacturing, the preferred molecular weight of a polyethylene glycol for derivatisation of a compound of the invention is from about 1 kDa to about 100 kDa, the term “about” indicating that in preparations of polyethylene glycol, some molecules will weigh more, some less, than the stated molecular weight. Polymers of other molecular weights may be used, depending on the desired therapeutic profile, for example the duration of sustained release desired, the effects, if any, on biological activity, the ease in handling, the degree or lack of antigenicity and other known effects of the polyethylene glycol to a therapeutic protein or analog. For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 45,000, 50,000, 55,000, 60,000, 65,000, 70,000, 75,000, 80,000, 85,000, 90,000, 95,000, or 100,000 kDa.

### Salts

Salt forms of compounds of the invention and of derivatives of such compounds also form part of the invention. In some embodiments the salt is a salt of a compound of the invention. In other embodiments the salt is a salt of a derivative of a compound of the invention.

Salts of compounds of the invention include those which are pharmaceutically acceptable, i.e. which are suitable for use in medicine. However, salts having non-pharmaceutically acceptable counterions are also within the scope of the present invention, for example, for use as intermediates in the preparation of the compounds.

Suitable salts according to the invention include those formed with organic or inorganic acids or bases. Pharmaceutically acceptable acid addition salts include those formed with hydrochloric, hydrobromic, sulphuric, nitric, citric, tartaric, acetic, phosphoric, lactic, pyruvic, acetic, trifluoroacetic, succinic, perchloric, fumaric, maleic, glycolic, salicylic, oxaloacetic, methanesulfonic, ethanesulfonic, p-toluenesulfonic, formic, benzoic, malonic, naphthalene-2-sulfonic, benzenesulfonic, and isethionic acids. Other acids such as oxalic acid may be useful as intermediates in obtaining the compounds of the invention in final form.

Pharmaceutically acceptable salts with bases include ammonium salts, alkali metal salts, for example potassium and sodium salts, alkaline earth metal salts, for example calcium and magnesium salts, and salts with organic bases, for example dicyclohexylamine and N-methyl-D-glucomine.

### Solvates

Those skilled in the art of organic and/or medicinal chemistry will appreciate that many organic compounds can form complexes with solvents in which they are reacted or from which they are precipitated or crystallized. Such complexes are known as “solvates”. For example, a complex with water is known as a “hydrate”. The invention also encompasses solvates of the compounds of the invention, solvates of derivatives of the compounds, and solvates of salts of the derivatives.

Those skilled in the art of organic and/or medicinal chemistry will also appreciate than many organic compounds can exist in different forms, including as amorphous material and/or in one or more crystalline forms. Different physical forms of organic compounds are known as polymorphs. The invention also encompasses all such different physical forms of the compounds of the invention, as well as different physical forms of their derivatives and salts.

### Biological Activity

Amylin appears to have at least three distinct receptor complexes able to bind to amylin with high affinity. Those receptors comprise the calcitonin receptor together with one of at least three alterative receptor activity modifying proteins (RAMPs)—RAMP1, RAMP2 or RAMP3 (Hay D L, Christopoulos G, Christopoulos A, Sexton P M (2004). Biochem. Soc. Trans. 32 (Pt 5): 865-7).

Compounds of the invention having activity at a human amylin receptor can be considered amylin receptor agonists. This may be assessed by, for example, an in vitro or cellular binding assay or by a reporter assay. Preferred compounds of the invention exhibit an activity at the human amylin receptor that is at least 1/50th that of human amylin, preferably an activity which is at least 1/30th, 1/20th, 1/10th, ⅕th, ⅓rd or ½that of human amylin, for example when tested in accordance with the assay described in the examples section below.

Methods of assessing activity at the amylin receptor are well known. For example, Homogeneous Time Resolved Fluorescence (Change C L et al., Activation of Calcitonin Receptor and Calcitonin Receptor-like Receptor by Membrane-anchored Ligands, Journal of Biological Chemistry Volume 285, Issue 2, 8 Jan. 2010, Pages 1075-1080) discloses a method of assaying for amylin receptor activation. A specific method is described in one f the examples herein.

Compounds of the invention fulfil some, or more preferably all, of the following criteria:
1) Sustained bioactivity at the human amylin receptor resulting in inhibition of appetite;
2) High solubility in aqueous solution at pH 3.8 to allow an effective dose to be administered in a low volume injection (thereby resulting in lower pain of injection). Solubility may be easily assessed by simple in vitro tests;
3) Long period of activity in vivo (as assessed in humans or an animal model) so as to permit injections no more frequently than daily and preferably no more than twice, or more preferably no more than once a week or more preferably no more than once a fortnight, whilst still producing acceptable therapeutic or cosmetic benefits;
4) Good weight loss (as assessed in human subjects or an animal model);

According to certain embodiments of the invention, especially embodiments relating to weight loss, obesity, carbohydrate metabolism and diabetes, the compounds, derivatives and salts of the invention have one, or both of the following features:
A. Sufficient solubility between pH 2 and pH 8 (for example between pH 4 and 8, or between pH 4 and pH7) to permit an effective dose to be administered in a volume of less than 1 ml, less than 0.5 ml or less than 0.3 ml;
B. One, several or all of the further 1 to 6 features listed above.

### Pharmacokinetics, Duration of Action and Solubility

Compounds of the present invention exhibit potent and prolonged duration of action in vivo following subcutaneous administration. In order to achieve this, the compounds are required to have both good activity at the biological target, and excellent pharmacokinetic properties.

Compounds of the present invention have a therapeutically useful duration of action and that manifests itself in the beneficial effects being observed in the experiments described herein below over several days. The half-life of compounds of the invention may be assessed in any suitable pharmacokinetic model, for example they may be assessed in a pig PK model. Preferred compounds of the invention were found to have a half-life significantly longer than human amylin. As well as exhibiting a long in vivo half-life, the compounds of the invention have good storage stability, with no significant degradation seen on storage in solution (for example sterile water or a sterile aqueous buffer solution) for 4 weeks at 4° C.

Poor water solubility is a known problem for lipid containing molecules. In contrast, the compounds of the invention have very good solubility.

### Conditions

The invention also provides a compound, derivative or salt of the invention, or a composition comprising the compound, derivative or salt together with a pharmaceutically acceptable carrier and optionally a further therapeutic agent, for use as a medicament.

The invention also provides a method of treating or preventing a disease or disorder or other non-desired physiological state in a subject comprising administration of a therapeutically effective amount of a compound, derivative or salt of the invention, or of a composition comprising the compound, derivative or salt together with a pharmaceutically acceptable carrier and optionally a further therapeutic agent. Preferably the compound, derivative, salt or composition is administered subcutaneously.

According to certain embodiments, the disease or disorder or other non-desired physiological state is diabetes or obesity, and particularly diabetes (e.g. type II diabetes).

According to certain embodiments, the disease or disorder or other non-desired physiological state may be the physiological state of being overweight.

The subject to whom the compound is administered may be overweight, for example, obese. Alternatively, or in addition, the subject may be diabetic, for example having insulin resistance or glucose intolerance, or both. The subject may have diabetes mellitus, for example, the subject may have Type II diabetes. The subject may be overweight, for example, obese and have diabetes mellitus, for example, Type II diabetes.

In addition, or alternatively, the subject may have, or may be at risk of having, a disorder in which obesity or being overweight is a risk factor. Such disorders include, but are not limited to, heart disease, cardiovascular disease, for example hypertension, atherosclerosis, congestive heart failure, and dyslipidemia; stroke; gallbladder disease; osteoarthritis; sleep apnoea; reproductive disorders for example, polycystic ovarian syndrome; cancers, for example breast, prostate, colon, endometrial, kidney, and esophagus cancer; varicose veins; acanthosis nigricans; eczema; exercise intolerance; insulin resistance; hypertension hypercholesterolemia; cholithiasis; osteoarthritis; orthopedic injury; insulin resistance, for example, type 2 diabetes and syndrome X; and thromboembolic disease (see Kopelman, Nature 404:635-43, 2000; Rissanen et al., British Med. J. 301, 835, 1990).

Other disorders associated with obesity include depression, anxiety, panic attacks, migraine headaches, PMS, chronic pain states, fibromyalgia, insomnia, impulsivity, obsessive compulsive disorder, and myoclonus. Certain neurological disorders and certain firms of neurological degeneration are also associated with obesity. Furthermore, obesity is a recognized risk factor for increased incidence of complications of general anesthesia (see e. g., Kopelman, Nature 404:635-43, 2000). In general, obesity reduces life span and carries a serious risk of co-morbidities such as those listed above.

Other diseases or disorders associated with obesity are birth defects, maternal obesity being associated with increased incidence of neural tube defects, carpal tunnel syndrome (CTS); chronic venous insufficiency (CVI); daytime sleepiness; deep vein thrombosis (DVT); end stage renal disease (ESRD); gout; heat disorders; impaired immune response; impaired respiratory function; infertility; liver disease; lower back pain; obstetric and gynecologic complications; pancreatitis; as well as abdominal hernias; acanthosis nigricans; endocrine abnormalities; chronic hypoxia and hypercapnia; dermatological effects; elephantitis; gastroesophageal reflux; heel spurs; lower extremity edema; mammegaly which causes considerable problems such as bra strap pain, skin damage, cervical pain, chronic odours and infections in the skin folds under the breasts, etc.; large anterior abdominal wall masses, for example abdominal panniculitis with frequent panniculitis, impeding walking, causing frequent infections, odours, clothing difficulties, low back pain; musculoskeletal disease; pseudotumor cerebri (or benign intracranial hypertension), and sliding hiatal hernia.

In some embodiments, the disease or disorder may be non-alcoholic fatty liver disease.

According to certain embodiments the disease or disorder or other non-desired physiological state may be being of a non-desired weight despite not being obese or overweight. The subject may be of normal weight (this includes but is not limited to subjects who were previously overweight or obese and who wish to prevent a return to an unhealthy weight). A subject may be a subject who desires weight loss, for example female and/or male subjects who desire a change in their appearance. In some cases where the subject is of a normal weight, aspects of the invention may relate to cosmetic treatment rather than to therapeutic treatment.

The invention also provides a method of reducing appetite in a subject, reducing food intake in a subject, reducing calorie intake in a subject, improving insulin release in a subject, improving carbohydrate metabolism in a subject, and/or improving carbohydrate tolerance in a subject, comprising administration of a therapeutically effective amount of a compound, derivative, salt or composition of the invention. Such methods may relate to treating subjects having a pre-diabetic state such as insulin insensitivity or pre-diabetes.

The invention also provides a method for improving a lipid profile in a subject comprising administration of a therapeutically effective amount of a compound, derivative, salt or composition of the invention. The invention also provides a method for alleviating a condition or disorder that can be alleviated by reducing nutrient availability comprising administration of a therapeutically effective amount of a compound, derivative, salt or composition of the invention.

A compound, derivative, salt or composition of the invention may be used for weight control and treatment, for example reduction or prevention of obesity, in particular any one or more of the following: preventing and reducing weight gain; inducing and promoting weight loss; and reducing obesity as measured by the Body Mass Index. A compound, derivative, salt or composition of the invention may be used in maintaining any one or more of a desired body weight, a desired Body Mass Index, a desired appearance and good health.

The present invention may also be used in treating, prevention, ameliorating or alleviating conditions or disorders caused by, complicated by, or aggravated by a relatively high nutrient availability. The term “condition or disorder which can be alleviated by reducing caloric (or nutrient) availability” is used herein to denote any condition or disorder in a subject that is either caused by, complicated by, or aggravated by a relatively high nutrient availability, or that can be alleviated by reducing nutrient availability, for example by decreasing food intake. Subjects who are insulin resistant, glucose intolerant, or have any form of diabetes mellitus, for example, type 1, 2 or gestational diabetes, can also benefit from methods in accordance with the present invention.

Conditions or disorders associated with increased caloric intake include, but are not limited to, insulin resistance, glucose intolerance, obesity, diabetes, including type 2 diabetes, eating disorders, insulin-resistance syndromes, and Alzheimer's disease.

The invention also provides a compound, derivative, salt or composition of the invention, for use in the treatment of obesity or diabetes.

The invention also provides a compound, derivative, salt or composition of the invention, for use in increasing energy expenditure of a subject, improving insulin release in a subject, improving carbohydrate tolerance in a subject and/or improving carbohydrate metabolism in a subject. Such use may relate to treating subjects having a pre-diabetic state such as insulin insensitivity or pre-diabetes.

The invention also provides a compound, derivative, salt or composition of the invention, for use in the reduction of appetite in a subject, use in the reduction of food intake in a subject, use in the reduction of calorie intake in a subject, use in improving insulin release in a subject, and/or use in improving carbohydrate tolerance in a subject. Such use may relate to treating subjects having a pre-diabetic state such as insulin insensitivity or pre-diabetes.

The invention also provides use of a compound, derivative, salt or composition of the invention for the manufacture of a medicament for the treatment of obesity or diabetes, of a subject, who may be as described above in reference to other aspects of the invention.

The invention also provides use of a compound, derivative or salt of the invention for the manufacture of a medicament for improving insulin release in a subject, for improving carbohydrate tolerance in a subject and/or improving carbohydrate metabolism in a subject. Such use may relate to treating subjects with a pre-diabetic state such as insulin insensitivity or pre-diabetes.

The invention also provides use of a compound, derivative or salt of the invention for the manufacture of a medicament for the reduction of appetite in a subject, reducing food intake in a subject, reducing calorie intake in a subject, improving insulin release in a subject, and/or use in improving carbohydrate tolerance in a subject.

According to certain embodiments the compound, derivative, salt or composition of the invention is to be administered parentally. According to other embodiments the compound, derivative, salt or composition of the invention is administered subcutaneously, intravenously, intramuscularly, intranasally, transdermally or sublingually. According to other embodiments the compound, derivative, salt or composition of the invention is administered orally. In one preferred embodiment compound, derivative, salt or composition of the invention is administered subcutaneously.

The compound, derivative, salt or composition of the invention is preferably used in the treatment of a human subject. However, while the compound, derivative, salt or composition of the invention will typically be used to treat human subjects they may also be used to treat similar or identical conditions in other vertebrates for example other primates; farm animals for example swine, cattle and poultry; sport animals for example horses; or companion animals for example dogs and cats.

### Compositions

It is preferable for the compound of the invention, or the derivative and/or the salt thereof, to be present in a pharmaceutical formulation or composition. Accordingly, the invention provides a composition comprising a compound, derivative or salt of the invention together with a pharmaceutically acceptable excipient and optionally another therapeutic ingredient. Compositions comprising the compound, derivative or salt are suitable for pharmaceutical use. According to certain preferred embodiments the composition is present in a syringe or other administration device for subcutaneous administration to humans. According to certain preferred embodiments the composition has a pH of less than 5. Compositions of the invention may take the form of a pharmaceutical formulation as described below.

The pharmaceutical formulations according to the invention include those suitable for oral, parenteral (including subcutaneous, intradermal, intramuscular, intravenous, and intra-articular), inhalation (including fine particle dusts or mists which may be generated by means of various types of metered dose pressurized aerosols, nebulizers or insufflators), rectal and topical (including dermal, transdermal, transmucosal, buccal, sublingual, and intraocular) administration, although the most suitable route may depend upon, for example, the condition and disorder of the recipient.

The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste. Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:2S, 1988, the contents of which are incorporated herein by reference.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein. The present compounds can, for example, be administered in a form suitable for immediate release or extended release. Immediate release or extended release can be achieved by the use of suitable pharmaceutical compositions comprising the present compounds or, particularly in the case of extended release, by the use of devices such as subcutaneous implants or osmotic pumps. The present compounds may also be administered liposomally.

Preferably, compositions according to the invention are suitable for subcutaneous administration, for example by injection. According to certain embodiments the composition may contain metal ions, for example copper, iron, aluminium, zinc, nickel or cobalt ions. The presence of such ions may limit solubility and thus delay absorption into the circulatory system from the site of subcutaneous administration.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate and/or lactose and/or other excipients, binders, extenders, disintegrants, diluents and lubricants such as those known in the art. Such compositions may also include a permeation enhancer. The compounds of the invention may also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxypropyl cellulose (HPC), hydroxypropyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor. An aqueous carrier may be, for example, an isotonic buffer solution at a pH of from about 3.0 to about 8.0, preferably at a pH of from about 3.5 to about 7.4, for example from 3.5 to 6.0, for example from 3.5 to about 5.0. Useful buffers include sodium citrate-citric acid and sodium phosphate-phosphoric acid, and sodium acetate/acetic acid buffers. The composition preferably does not include any compounds known to be deleterious to peptide compounds.

Excipients that can be included are, for instance, other proteins, such as human serum albumin or plasma preparations. If desired, the pharmaceutical composition may also contain minor amounts of non-toxic auxiliary substances, such as wetting or emulsifying agents, preservatives, and pH buffering agents and the like, for example sodium acetate or sorbitan monolaurate.

Exemplary compositions for nasal aerosol or inhalation administration include solutions in saline, which can contain, for example, benzyl alcohol or other suitable preservatives, absorption promoters to enhance bioavailability, and/or other solubilizing or dispersing agents such as those known in the art. Conveniently in compositions for nasal aerosol or inhalation administration the compound of the invention is delivered in the form of an aerosol spray presentation from a pressurized pack or a nebulizer, with the use of a suitable propellant, e.g., dichlorodifluoro-methane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated to contain a powder mix of the compound and a suitable powder base, for example lactose or starch. In one specific, non-limiting example, a compound of the invention is administered as an aerosol from a metered dose valve, through an aerosol adapter also known as an actuator. Optionally, a stabilizer is also included, and/or porous particles for deep lung delivery are included (e.g., see U.S. Pat. No. 6,447,743).

Formulations for rectal administration may be presented as a retention enema or a suppository with the usual carriers such as cocoa butter, synthetic glyceride esters or polyethylene glycol. Such carriers are typically solid at ordinary temperatures, but liquefy and/or dissolve in the rectal cavity to release the drug.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia.

Exemplary compositions for topical administration include a topical carrier such as Plastibase (mineral oil gelled with polyethylene).

Preferred unit dosage formulations are those containing an effective dose, as hereinbefore recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavouring agents.

The compounds, derivatives and salts of the invention may also be suitably administered as sustained release systems. Suitable examples of sustained release systems of the invention include suitable polymeric materials, for example semi permeable polymer matrices in the form of shaped articles, e.g., films, or microcapsules; suitable hydrophobic materials, for example as an emulsion in an acceptable oil; or ion exchange resins; and sparingly soluble derivatives of the compound of the invention, for example, a sparingly soluble salt. Sustained release systems may be administered orally; rectally; parenterally; intracisternally; intravaginally; intraperitoneally; topically, for example as a powder, ointment, gel, drop or transdermal patch; bucally; or as an oral or nasal spray.

Preparations for administration can be suitably formulated to give controlled release of compounds, derivatives and salts of the invention. For example, the pharmaceutical compositions may be in the form of particles comprising one or more of biodegradable polymers, polysaccharide jellifying and/or bioadhesive polymers, amphiphilic polymers, agents capable of modifying the interface properties of particles of the compounds of the invention. These compositions exhibit certain biocompatibility features which allow a controlled release of the active substance, see U.S. Pat. No. 5,700,486, the contents of which is incorporated herein by reference.

The use of a controlled release composition is preferred for indications such as the treatment of obesity and/or diabetes, where maximising the time period between injections is desirable. However, for indications such as providing neuroprotection or cardiac protection (e.g. following suspected myocardial infarction or stroke), where it is desired to achieve a therapeutic plasma concentration of the active agent in as short a time period as possible, an immediate release formulation will be preferred. In such cases, a dosage regime comprising administration of a dose of an immediate release formulation of the active agent (i.e. as soon as possible after suspected myocardial infarction or stroke) and subsequent administration of a dose of a controlled release formulation of the active agent may be preferred.

A compound, derivative or salt of the invention may be delivered by way of a pump (see Langer, supra; Sefton, CRC Crit. Ref. Biomed. Eng. 14:201, 1987; Buchwald et al., Surgery 88:507, 1980; Saudek et al., N. Engl. J. Med. 321:574, 1989) or by a continuous subcutaneous infusion, for example, using a mini-pump. An intravenous bag solution may also be employed. The key factor in selecting an appropriate dose is the result obtained, as measured by decreases in total body weight or ratio of fat to lean mass, or by other criteria for measuring control or prevention of obesity or prevention of obesity-related conditions, as are deemed appropriate by the practitioner. Other controlled release systems are discussed in the review by Langer (Science 249:1527-1533, 1990) which is incorporated herein by reference. In another aspect of the disclosure compounds of the invention are delivered by way of an implanted pump, described, for example, in U.S. Pat. Nos. 6,436,091; 5,939,380; 5,993,414, the contents of which are incorporated herein by reference.

Implantable drug infusion devices are used to provide patients with a constant and long-term dosage or infusion of a drug or any other therapeutic agent. Essentially such device may be categorized as either active or passive. A compound, derivative or salt of the present invention may be formulated as a depot preparation. Such a long acting depot formulation can be administered by implantation, for example subcutaneously or intramuscularly; or by intramuscular injection. Thus, for example, the active ingredient can be formulated with suitable polymeric or hydrophobic materials, for example as an emulsion in an acceptable oil; or ion exchange resins; or as a sparingly soluble derivatives, for example, as a sparingly soluble salt.

A therapeutically effective amount of the active agent of the invention may be administered as a single pulse dose, as a bolus dose, or as pulse doses administered over time. Thus, in pulse doses, a bolus administration of the active agent is provided, followed by a time period wherein no active agent is administered to the subject, followed by a second bolus administration. In specific, non-limiting examples, pulse doses are administered during the course of a day, during the course of a week, or during the course of a month.

### Combination Treatments

In certain embodiments, a therapeutically effective amount of a compound, derivative, salt or composition of the invention is administered with a therapeutically effective amount of a further agent or agents. The compound, derivative or salt may for example be administered simultaneously with one or more further therapeutic agent(s), or it may be administered sequentially or separately. Accordingly, the invention provides a compound, derivative or salt of the invention for use as a medicament, wherein the compound, derivative or salt is for use with a therapeutically effective amount of a further therapeutic agent or agents (e.g. for administration simultaneously, sequentially or separately). In certain embodiments, the active agent of the invention is formulated and administered with a further therapeutic agent or agents as a single dose.

In certain embodiments, the further therapeutic agent or agents is/are an additional anti-diabetic, appetite suppressant, a food-intake-reducing, plasma glucose-lowering or plasma lipid-altering agent. Specific, non-limiting examples of an additional appetite suppressant include cagrilitide, Pramlintide amfepramone (diethylpropion), phentermine, mazindol and phenylpropanolamine, fenfluramine, dexfenfluramine, phendimetrazine, benzphetamine, sibutramine, rimonabant, topiramate, fluoxetine, bupropion, zonisamide, naltrexone, orlistat and cetilistat. Specific, non-limiting examples of an additional anti-diabetic agent include metformin, phenformin, rosiglitazone, pioglitazone, troglitazone, repaglinide, nateglinide, tolbutamide, acetohexamide, tolazamide, chlorpropamide, glipizide, glyburide, glimepiride, gliclazide, fibroblast growth factor 21, miglitol, acarbose, exenatide, pramlintide, vildagliptin and sitagliptin.

In a preferred embodiment, the further therapeutic agent or agents is/are GLP-1 or a GLP-1 derivative, analogue or agonist.

In alternative embodiments, the further therapeutic agent or agents is/are insulin or an insulin analogue, derivative or agonist.

In alternative embodiments, the further therapeutic agent or agents is/are GIP or a GIP analogue, derivative or agonist.

In alternative embodiments, the further therapeutic agent or agents is/are glucagon or a glucagon analogue, derivative or agonist.

### Dosages

A compound, derivative, salt or composition of the invention may be administered whenever the effect, e.g., appetite suppression, decreased food intake or decreased caloric intake, is desired, or slightly before to whenever the effect is desired, such as, but not limited to, about 10 minutes, about 15 minutes, about 30 minutes, about 60 minutes, about 90 minutes, or about 120 minutes, before the time the effect is desired.

Alternatively or additionally, a compound, derivative, salt or composition of the invention may be administered on a regular dosage regime. For example, once a day, once a week, once every 10 day or once every 2 weeks.

The therapeutically effective amount of the active agent of the invention will be dependent on the molecule utilized, the subject being treated, the severity and type of the affliction, and the manner and route of administration. For example, a therapeutically effective amount of a compound of the invention may vary from about 0.01 μg per kilogram (kg) body weight to about 1 g per kg body weight, for example about 0.1 μg to about 20 mg per kg body weight, for example about 1 μg to about 5 mg per kg body weight, or about 5 μg to about 1 mg per kg body weight. When used in a combination therapy or composition alongside another therapeutic agent, the dosage of the active agent of the invention may optionally be half the dosage listed above.

In one embodiment of the invention, a compound, derivative or salt of the invention may be administered to a subject at from 0.5 to 1,333 nmol per kg bodyweight, for example 1 to 1,333 nmol/kg bodyweight, for example 2 to 1,000 nmol per kg bodyweight, for example 4 to 1,333 nmol per kg bodyweight, for example from 5 to 1,000 nmol per kg bodyweight, for example at from 10 to 750 nmol per kg bodyweight, for example at from 20 to 500 nmol per kg bodyweight, in particular at from 30 to 240 nmol per kg bodyweight. In a preferred embodiment, a high activity compound of the invention is administered to a subject at from 0.2 to 10 nmol per kg bodyweight, for example 0.5 to 5.0 nmol/kg bodyweight, for example 1.0 to 2.0 nmol per kg bodyweight, for example 1.5 nmol per kg bodyweight., For a 75 kg subject, such doses correspond to dosages of from 37.5 nmol to 100 μmol, for example from 75 nmol to 100 μmol, for example from 150 nmol to 100 μmol, for example from 300 nmol to 100 μmol, for example from 375 nmol to 75 μmol, for example from 750 nmol to 56.25 μmol, for example from 1.5 to 37.5 μmol, in particular from 2.25 to 18 μmol. In a preferred embodiment for a high activity compound of the invention, for a 75 kg subject, such doses correspond to dosages of from 15 to 750 nmol, for example 37.5 to 375.0 nmol, for example 75 to 150 nmol, for example 112.5 nmol. The invention also contemplates dosages ranges bounded by any of the specific dosages mentioned herein.

The exact dose is readily determined by one of skill in the art based on the potency of the specific compound utilized, the route of delivery of the compound and the age, weight, sex and physiological condition of the subject.

For a compound with a long blood half-life, the doses discussed above may be given, for example, once or twice per month, or once, twice, three-times or four-times per week. For a preferred compound, a dose may be given no more frequently than once a week. Alternatively, for a compound with a shorter half-life in the blood, the doses discussed above may be given, for example, once, twice, three-times or four-times a day or once or twice a week. In some embodiments, a dose may be given once every 2, 3 or 4 days. According to certain embodiments they may be administered once shortly before each meal to be taken.

### Examples

The invention is further described with reference to the following non-limiting examples.

### Materials and Methods

### Peptide Synthesis

Peptide synthesis was carried out on a Rink amide MBHA resin. Amino acids were attached using the Fmoc strategy. For the main peptide portion of the molecule, each amino acid was added sequentially from the C- to the N-termini. Peptide couplings were mediated using reagents such as HBTU. Peptide cleavage from the resin was achieved with trifluoracetic acid in the presence of scavengers. In a second stage, the glutamic acid residue to be added to the N-terminus of the main peptide portion was substituted was functionalised with the fatty dicarboxylic acid group before being attached via one or other of its carboxylic acid groups.

Peptides were purified by reverse phase HPLC. Quality control was performed on all purified peptides and peptides were shown in most cases to be greater than 90% pure by HPLC in two buffer systems. MALDI-MS showed the expected molecular ion.

### Example Synthesis

Example compound A233 was prepared as follows using standard Fmoc chemistry:
1) Resin preparation: Rink Amide MBHA Resin (0.6 mmol, 1.00 eq, Sub 0.3 mmol/g) in DMF was agitated with N₂ for 2 hrs at 20° C. Then the mixture was filtered to get the resin.
2) Deprotection: 20% piperidine in DMF (25.00 mL) was added to the resin and the mixture was agitated with N₂ for 15 min at 20° C. The resin was washed with DMF (5 washes of 25.00 mL) and filtered to obtain the resin.
3) Coupling: A solution of HBTU (648 mg, 1.71 mmol, 2.85 eq) and Fmoc-Pro-OH (606 mg, 1.8 mmol, 3.00 eq) in DMF (10.0 mL) was added with DIPEA (N,N-Diisopropylethylamine) (0.63 ml, 3.6 mmol, 6.00 eq) to the resin and agitated with N₂ for 30 min at 20° C. The resin was then washed five times with DMF (25.0 mL
4) Steps 3 and 4 were repeated for the coupling of subsequent amino acids.

### Note:

| # | Materials | Coupling reagents |
|---|---|---|
| 2 | Fmoc-Thr(tBu)—OH (3.00 eq) | HBTU (2.85 eq) and DIPEA (6.00 eq) |
| 3 | Fmoc-Asn(Trt)—OH (3.00 eq) | HBTU (2.85 eq) and DIPEA (6.00 eq) |
| 4 | Fmoc-Ser(tBu)—OH (3.00 eq) | HBTU (2.85 eq) and DIPEA (6.00 eq) |
| 5 | Fmoc-Gly—OH (3.00 eq) | HBTU (2.85 eq) and DIPEA (6.00 eq) |
| 6 | Fmoc-Val—OH (3.00 eq) | HBTU (2.85 eq) and DIPEA (6.00 eq) |
| 7 | Fmoc-Pro—OH (3.00 eq) | HBTU (2.85 eq) and DIPEA (6.00 eq) |
| 8 | Fmoc-Thr(tBu)—OH (3.00 eq) | HBTU (2.85 eq) and DIPEA (6.00 eq) |
| 9 | Fmoc-Arg(Pbf)—OH (3.00 eq) | HBTU (2.85 eq) and DIPEA (6.00 eq) |
| 10 | Fmoc-Pro—OH (3.00 eq) | HBTU (2.85 eq) and DIPEA (6.00 eq) |
| 11 | Fmoc-Tyr(tBu)—OH (3.00 eq) | HBTU (2.85 eq) and DIPEA (6.00 eq) |
| 12 | Fmoc-Thr(tBu)—OH (3.00 eq) | HBTU (2.85 eq) and DIPEA (6.00 eq) |
| 13 | Fmoc-Gln(Trt)—OH (3.00 eq) | HBTU (2.85 eq) and DIPEA (6.00 eq) |
| 14 | Fmoc-Leu—OH (3.00 eq) | HBTU (2.85 eq) and DIPEA (6.00 eq) |
| 15 | Fmoc-Lys(Boc)—OH (3.00 eq) | HBTU (2.85 eq) and DIPEA (6.00 eq) |
| 16 | Fmoc-His(Trt)—OH (3.00 eq) | HBTU (2.85 eq) and DIPEA (6.00 eq) |
| 17 | Fmoc-Leu—OH (3.00 eq) | HBTU (2.85 eq) and DIPEA (6.00 eq) |
| 18 | Fmoc-Glu(OtBu)--OH (3.00 eq) | HBTU (2.85 eq) and DIPEA (6.00 eq) |
| 19 | Fmoc-Glu(OtBu)--OH (3.00 eq) | HBTU (2.85 eq) and DIPEA (6.00 eq) |
| 20 | Fmoc-Ala—OH (3.00 eq) | HBTU (2.85 eq) and DIPEA (6.00 eq) |
| 21 | Fmoc-Leu—OH (3.00 eq) | HBTU (2.85 eq) and DIPEA (6.00 eq) |
| 22 | Fmoc-Arg(Pbf)—OH (3.00 eq) | HBTU (2.85 eq) and DIPEA (6.00 eq) |
| 23 | Fmoc-Gln(Trt)—OH (3.00 eq) | HBTU (2.85 eq) and DIPEA (6.00 eq) |
| 24 | Fmoc-Thr(tBu)—OH (3.00 eq) | HATU (2.85 eq) and DIPEA (6.00 eq) |
| 25 | Fmoc-Ala—OH (3.00 eq) | HATU (2.85 eq) and DIPEA (6.00 eq) |
| 26 | Fmoc-Cys(Trt)—OH (3.00 eq) | HATU (2.85 eq) and DIPEA (6.00 eq) |
| 27 | Fmoc-Thr(tBu)—OH (3.00 eq) | HATU (2.85 eq) and DIPEA (6.00 eq) |
| 28 | Fmoc-Ala—OH (3.00 eq) | HATU (2.85 eq) and DIPEA (6.00 eq) |
| 29 | Fmoc-Thr(tBu)—OH (3.00 eq) | HATU (2.85 eq) and DIPEA (6.00 eq) |
| 30 | Fmoc-Ser(tBu)—OH (3.00 eq) | HATU (2.85 eq) and DIPEA (6.00 eq) |
| 31 | Fmoc-Cys(Trt)—OH (3.00 eq) | HATU (2.85 eq) and DIPEA (6.00 eq) |
| 32 | Fmoc-Lys(Boc)—OH (3.00 eq) | HATU (2.85 eq) and DIPEA (6.00 eq) |
| 33 | Fmoc-Lys(Boc)—OH (3.00 eq) | HATU (2.85 eq) and DIPEA (6.00 eq) |
| 34 | Fmoc-Glu-OtBu (3.00 eq) | HATU (2.85 eq) and DIPEA (6.00 eq) |
| 35 | 20-(tert-butoxy)-20-oxoicosanoic acid (3.00 eq) | HATU (2.85 eq) and DIPEA (6.00 eq) |

* As the protected glutamic acid reagent 34 has its C-1 acid group protected with TBU, it reacts at its C-5 acid with the lysine 33.

### Peptide Cleavage, Disulphide Bond Formation and Purification:

5) The resin was washed twice with MeOH (20.0 mL) and dried under vacuum to provide 8.0 g peptide resin. Then 80 mL of cleavage buffer (92.5% TFA/2.5% Mpr/2.5% TIS/2.5% H₂O) was added to the flask containing the side chain protected peptide resin at 20° C. and the mixture was stirred for 2.5 h. The peptide was precipitated with cold tert-butyl methyl ether (800 mL) and centrifuged (2 min at 3000 rpm). The peptide precipitate was washed with tert-butyl methyl ether for two more times (400 mL). The crude peptide was dried and its identity confirmed by LCMS.
6) To the crude peptide (1.32 g) in water (150 mL) and CH₃CN (100.0 mL) was added I₂ in MeOH (0.10 M) dropwise at 20° C. until a yellow color persisted. Then the mixture was stirred at 20° C. for 2 min. After 2 min, sodium thiosulfate (0.10 M in water) was added dropwise until the yellow color disappeared. The mixture was lyophilized to give a crude powder.
7) The residue was purified by prep-HPLC (TFA condition; 30° C., A:0.075% TFA/H₂O, B:CH₃CN) to give the example compound (166.9 mg, 40.8 μmol, 6.8% yield, 92.94% purity, TFA) as a white solid the identity of which was confirmed by LCMS).

Equivalent methods were employed for all of the other peptides described herein.

### Receptor Potency of Peptides at the Human Amylin Receptor.

Biological activity was assessed by potency of peptides to stimulate cAMP production in human embryo kidney cells with a tetracycline-regulated mammalian expression system (T-Rex-293, Invitrogen) cell lines overexpressing the human amylin receptor 3 (human calcitonin receptor and receptor activity modifying protein 3). Cells were transfected with the human calcitonin receptor plasmid and RAMP3 plasmid (Azenta Life Sciences) 24 h prior to treatment with compounds. Individual wells of cells receive a concentration of test item, and a dose response of each compound is tested. A commercial cAMP kit (Cisbio) was used to quantify cAMP in the cell via HTRF (Homogeneous Time-Resolved Fluorescence) technology after 30 mins of peptide stimulation and a further 1 h lysis. Plates were read on a SpectraMax i3x Multi-Mode Detection Platform plate reader and concentration response curves drawn with Graph Pad Prism 8.0 (or higher). EC₅₀ values were generated for each peptide and compared to the controls for the day.

### In Vivo Efficacy Study: Single Dose Feeding Studies in Male Rats

Male rats (Charles River Ltd, Margate, UK) were used for animal experiments. Ad libitum fed rats were individually housed in IVC cages. Animals were randomised into treatment groups, with stratification by body weight. Peptide solutions were prepared freshly immediately prior to administration. The control animals were dosed water 5% v/v water and 95% NaCl (0.9% w/v) whilst peptides (either 1.5 or 15 nmol/kg body weight) were resuspended in water for injection. Peptide and vehicle were administered in the early light phase (0900-1000) by subcutaneous injection and animals provided a known amount of food. Animals were given free access to food and water during the study period. Animal body weight and remaining food were weighed throughout the study, typically 24, 48, 72 and 96, and in some examples 168h post dosing. The results presented are the figures 3 days post dosing.

The compounds were assessed for their propensity to inhibit food intake and their ability to bring about body weight change, reported as two values, called “potency” and “food” in FIG. 1. For “potency”, a score is taken by 2× the difference between weight change, measured in grams, in the treatment group and vehicle control group measured at 3 days post dose. For “food”, a score is taken by the difference in food intake, measured in grams, consumed by the treatment group and vehicle control group measured at 3 days post dose.

1. A compound having the formula A-B-C, or a derivative thereof, or a salt or solvate of any of the foregoing, wherein:
A is Q, Q-Ser, Q-Glu, or Q-Lys, wherein Q is a group of the formula: 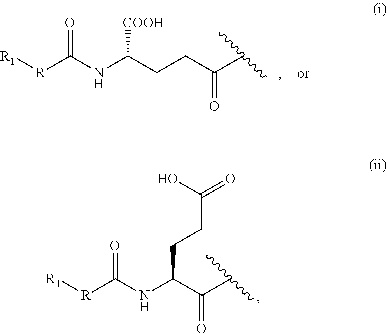
wherein R is a C₈-C₂₈ alkylene or alkenylene chain and R₁ is —CO₂H;
B is a peptide moiety of 32 or 33 amino acid residues, having a sequence of X-Y-Z, wherein:
X is Lys-Cys;
Y is Xaa1-Xaa2-Xaa3-Xaa4-Xaa5-Xaa6, wherein:
Xaa1 is Ser or absent;
Xaa2 is Asn, Lys, Ser, or Thr;
Xaa3 is Thr or Leu;
Xaa4 is Ala or Ser;
Xaa5 is Thr; and
Xaa6 is Cys;
and
Z is Xaa7-Xaa8-Xaa9-Xaa10-Xaa11-Xaa12-Xaa13-Xaa14-Xaa15-Xaa16-Xaa17-Xaa18-Xaa19-Xaa20-Xaa21-Xaa22-Xaa23-Xaa24-Xaa25-Xaa26-Xaa27-Xaa28-Xaa29-Xaa30-Xaa31 (SEQ ID. NO.: 6), wherein:
Xaa7 is Ala, Val, or Met;
Xaa8 is Thr or Leu;
Xaa9 is Gln or Gly;
Xaa10 is Arg or Lys;
Xaa11 is Leu;
Xaa12 is Ala or Ser;
Xaa13 is any amino acid;
Xaa14 is Glu, Asp, or Phe;
Xaa15 is Leu;
Xaa16 is His;
Xaa17 is Lys or Arg;
Xaa18 is Leu;
Xaa19 is Gln or Lys;
Xaa20 is Thr;
Xaa21 is Tyr or Phe;
Xaa22 is Pro;
Xaa23 is Arg or Lys;
Xaa24 is Thr;
Xaa25 is Gln, Asp, Pro, Lys, or Asn;
Xaa26 is Val or Thr;
Xaa27 is Gly;
Xaa28 is Ser or Ala;
Xaa29 is Lys, Asn, Gly, or Asp;
Xaa30 is Thr, or Ala; and
Xaa31 is any amino acid;
and
C is a terminal —NH₂ group bound to the C terminal of the peptide moiety B.

2. The compound of claim 1, or the derivative thereof, or the salt or solvate of any of the foregoing, wherein there exists a disulphide bridge between the side chains of the two Cys residues.

3. The compound of claim 1, or the derivative thereof, or the salt or solvate of any of the foregoing, wherein Xaa1 is absent.

4. The compound of claim 3, or the derivative thereof, or the salt or solvate of any of the foregoing, wherein Xaa2 is Asn, Lys, or Ser; Xaa3 Thr; and Xaa4 is Ala.

5. The compound of claim 3, or the derivative thereof, or the salt or solvate of any of the foregoing, wherein Xaa7 is Ala; Xaa8 is Thr; Xaa9 is Gln; Xaa10 is Arg; and Xaa12 is Ala.

6. The compound of claim 3, or the derivative thereof, or the salt or solvate of any of the foregoing, wherein Xaa13 is Glu, Asp, Gln, Asn, or Lys; Xaa14 is Glu; Xaa15 is Leu; Xaa17 is Lys, His, or Arg; Xaa18 is Leu; Xaa19 is Gln; Xaa22 is Pro; and Xaa24 is Thr.

7. The compound of claim 6, or the derivative thereof, or the salt or solvate of any of the foregoing, wherein Xaa13 is Asp, Glu, or Gln.

8. The compound of claim 6, or the derivative thereof, or the salt or solvate of any of the foregoing, wherein Xaa17 is Lys.

9. The compound of claim 3, or the derivative thereof, or the salt or solvate of any of the foregoing, wherein Xaa25 is Pro; Xaa26 is Val; Xaa27 is Gly; and Xaa28 is Ser.

10. The compound of claim 3, or the derivative thereof, or the salt or solvate of any of the foregoing, wherein Xaa29 is Gly, and Xaa30 is Thr or Ala; or wherein Xaa29 is Asn, and Xaa30 is Thr or Ala.

11. (canceled)

12. The compound of claim 10, or the derivative thereof, or the salt or solvate of any of the foregoing, wherein Xaa30 is Thr; and Xaa31 is Pro.

13. The compound of claim 1, or the derivative thereof, or the salt or solvate of any of the foregoing, comprising an amino acid sequence corresponding to any one of the amino acid sequences of SEQ ID NOs: 12-146.

14. The compound of claim 1, or the derivative thereof, or the salt or solvate of any of the foregoing, wherein:
A is Q-Lys, wherein Q is a group of the formula: 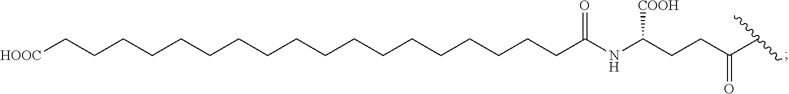 [SMILES: *C(=O)CC[C@H](NC(=O)CCCCCCCCCCCCCCCCCCC(=O)O)C(=O)O]
B is a peptide moiety of 32 amino acid residues, having the sequence Lys-Cys-Ser-Thr-Ala-Thr-Cys-Ala-Thr-Gln-Arg-Leu-Ala-Glu-Glu-Leu-His-Lys-Leu-Gln-Thr-Tyr-Pro-Arg-Thr-Pro-Val-Gly-Ser-Asn-Thr-Pro;
and
C is a terminal —NH₂ group bound to the C terminal of the peptide moiety B.

15. The compound of claim 1, or the derivative thereof, or the salt or solvate of any of the foregoing, wherein R has an even number of carbon atoms.

16. The compound of claim 15, or the derivative thereof, or the salt or solvate of any of the foregoing, wherein R is a C₁₆ or C₁₈ alkylene or alkenylene group.

17. The compound of claim 16, or the derivative thereof, or the salt or solvate of any of the foregoing, wherein R is a C₁₈ straight chain alkylene group.

18. The compound of claim 1, or the derivative thereof, or the salt or solvate of any of the foregoing wherein the derivative comprises one or more derivatisations selected from amidation, glycosylation, carbamylation, acylation, sulfation, phosphorylation, cyclization, lipidization, pegylation, and fusion to another peptide or protein to form a fusion protein.

19. The compound of claim 1, or the derivative thereof, or the salt or solvate of any of the foregoing together with a further therapeutic agent, for simultaneous, sequential or separate administration.

20. The compound of claim 19, or the derivative thereof, or the salt or solvate of any of the foregoing, wherein the further therapeutic agent is a GLP-1 receptor agonist, a Y2 (PYY) receptor agonist, SIP receptor agonist, or a glucagon receptor agonist.

21. The compound of claim 19, or the derivative thereof, or the salt or solvate of any of the foregoing, wherein the further therapeutic agent is insulin or an insulin derivative or agonist, GLP-1 or a GLP-1 derivative or agonist, GIP or a GIP derivative or agonist, or glucagon or a glucagon derivative or agonist.

22. A composition comprising the compound of claim 1, or the derivative thereof, or the salt or solvate of any of the foregoing, together with a pharmaceutically acceptable carrier.

23. The composition of claim 22, present in a syringe or other administration device for subcutaneous administration to humans.

24. (canceled)

25. A method of treating or preventing a disease or disorder or other non-desired physiological state in a subject comprising administering to the subject a therapeutically effective amount of the compound of claim 1, or the derivative thereof, or the salt or solvate of any of the foregoing.

26. (canceled)

27. A method of treating or preventing diabetes, obesity, heart disease, stroke or non-alcoholic fatty liver disease in a subject, improving insulin release in a subject, improving carbohydrate metabolism in a subject, improving the lipid profile of a subject, improving carbohydrate tolerance in a subject, reducing appetite, reducing food intake, and/or reducing calorie intake, comprising administering to the subject a therapeutically effective amount of the compound of claim 1, or the derivative thereof, or the salt or solvate of any of the foregoing.

28. (canceled)

29. A method of causing weight loss or preventing weight gain in a subject for cosmetic purposes comprising administering to the subject an effective amount of the compound of claim 1, or the derivative thereof, or the salt or solvate of any of the foregoing.
